(19) United States
(12) Patent Application Publication
(10) Pub. No.: US 2004/0023864 A1
(43) Pub. Date: Feb. 5, 2004

(54) Method of regulating angiogenesis using ryk protein

(76) Inventors: Steve Roczniak, Lafayette, CA (US); Nathalie A. Dubois-Stringfellow, Berkeley, CA (US); Alya Zolotorev, Oakland, CA (US)

Correspondence Address: Bayer Corporation, 800 Dwight Way, PO Box 1986, Berkeley, CA, 94701, US

(21) Appl. No.: 10/275,589

(22) PCT Filed: May 9, 2001
(86) PCT No.: PCT/US01/15043

(51) Int. Cl. A61K 38/17

(52) U.S. Cl. 514/12

(57) ABSTRACT

Ryk protein was found to have a novel activity in regulating angiogenesis. New variant Ryk proteins were constructed that were useful in modulating the capillary forming activity of endothelial cells. Variant Ryk proteins, may be employed as therpeutics in diseases such as cancer, wound healing, diabetic retinopathies, macular degeneration, and cardiovascular diseases, and other diseases or clinical conditions where angiogenesis is relevant to the causation or treatment of the disease.

### BACKGROUND OF THE INVENTION

1. Field

This invention relates to a method of regulating angiogenesis using newly identified polypeptides or polynucleotides encoding such polypeptides. More particularly, the method comprises using the Ryk protein, or fragments derived therefrom, to regulate angiogenesis in human or animal tissue.

2. Background

Ryk is a member of the Receptor Tyrosine Kinase (RK) family that has impaired catalytic activity and whose ligand(s) have not yet been identified. Ryk has been described by Stacker, et al. in WIPO publication WO 93/23429 (Appl'n No. PCT/AU93/00210). The open reading frame of human Ryk encodes 607 amino acids (aa), has 2 potential trannembrane domains and exhibits closest homology in its catalytic (i.e., activation and nucleotide binding) domain to RTKs such as met (HGF/SF-R) and IGP-1R (Tamagnone et al., Oncogene 1993; 8:2009). The Ryk catalytic region shows closest homology with v-sea (39%) (Wang et al., Mol. Med. 1996; 2:189). Ryk differs from other RTK family members at a number of conserved residues in its catalytic domain (Katso et al., Mol. Cell. Biol. 1999; 19:6427). The amino acid substitutions in this domain account for Ryk's inability to undergo autophosphorylation or to phosphorylate substrates. However, ligand stimulation of a Ryk chimeric receptor results in activation of mitogen-activated protein kinase pathway (Katso et al., Mol. Cell. Biol. 1999; 19:6427).

Ryk does not exhibit significant homology in its extracellular domain to other RTK families. Ryk has a relatively small extracellular domain. Variations of the amino acid/nucleotide sequence of Ryk has been described by several authors (See e.g., Tamagnone et al., Oncogene 1993; 8:2009 (the extracellular domain is described as having an amino acid sequence consisting of 191 amino acids) and Halford et al., J. Biol. Chem. 1999; 274:7379 (the extracellular domain is described as having the same amino acid sequence as described by Tamagnone; however, ten amino acids at the C terminus are missing making the extracellular domain 181 amino acids)). All of the extracellular domains of Ryk described in the literature, however, contain five potential N-linked glycosylation sites (Stacker et al., PNAS 1992; 89:11818). The human and mouse ryk sequences are 92% identical at the nucleotide level and 97% identical at the amino acid level (Stacker et al., PNAS 1992; 89:11818).

Immunochemical studies of normal tissues have indicated that Ryk is expressed in epithelium, stroma and blood vessels (Katso et al., Cancer Res. 1999; 59:2265). Northern Blot analyses show the presence of the Ryk mRNA in many tissues, including heart, brain, lung, placenta, liver, muscle, kidney and pancreas (Wang et al., Mol. Mode 1996; 2:189). In situ hybridization analyses have shown that the Ryk gene is expressed almost exclusively in the epithelial and stromal compartments of the brain, lung, colon, kidney and breast (Wang et al., Mol. Med. 1996; 2:189). Ryk RNA is present at greatly increased levels in the basal layer of skin and tongue epithelia and the intervillous layer and some crypt bases of the intestine (Serfas et al., Oncogene 1998; 17:3435). Ryk is induced in epithelial cells seeking a final place in a differentiated tissue or during remodeling of the endometrium, and it has been hypothesized to be involved in cellular recognition (Serfas et al., Oncogene 1998; 17:3435).

Human Ryk is overexpressed in borderline and malignant ovarian tumors (Katso et al., Cancer Res. 1999; 59:2265). In serous and clear cell tumor subtypes, increased expression is observed in epithelium, stroma, and blood vessels. In malignant tumors, the increased expression is predominantly confined to epithelium (Wang et al., Mol. Med 1996; 2:189). The human Ryk cDNA has been isolated from a complementary DNA library of SKOV-3, an epithelial ovarian cancer cell line, using PCR (Wang et al., Mol. Med. 1996; 2:189). There is minimal to absent expression of human Ryk on the surface epithelium of normal ovaries. Overexpression of human Ryk in the mouse fibroblast cell line NIH3T3 induces anchorage-independent growth and tumorigenicity in nude mice (Katso et al., Cancer Res. 1999; 59:2265). These observations suggest that Ryk may be involved in tumor progression.

RYK is the mammalian homologue of Drosophila Lio protein which has been determined to be involved in learning and memory and axon guidance (pathway selection) in the embryo & adult (Moreau-Fauvarque et al., Mech. Dev. 1998; 78:47). Lionette was identified in screens for embryonic nervous system axonal guidance defects called derailed. Lionette mutants (derailed) are viable but present structual brain defects in the adult central complex where the central brain axons behave as if abnormally attracted by midbrain area. In derailed mutant embryos, a small set of interneurons that expressed the gene failed to make correct pathway choices (Callahan et al. Nature 1995; 376:171).

Ryk is expressed in CD3-, CD4-, and CD8-T cells, pre-T cells, thymic epithelial cells, and mature myeloid cells, but not myeloid precursors or B cell precursors (Simoneaux et al., J. Immunol. 1995; 154:1157). Ryk expression is observed in differentiated cells (Lin+) but not in precursor cells (Lin−), and it was hypothesized that Ryk expression may be regulated during hematopoietic development by lineage commitment and stage of maturation.

Ryk is also expressed by a small subset of developing embryonic muscles and neighboring epidermal cells during muscle attachment site selection (Callahan et al., Development 1996; 122:2761). In derailed mutants, muscles often fail to attach at appropriate locations although their epidermal attachment cells appear unaffected.

Angiogenesis, the formation of new capillaries from preexisting blood vessels, is a multistep, highly orchestrated process involving vessel sprouting, endothelial cell migration, proliferation, tube differentiation, and survival. Several lines of direct evidence now suggest that angiogenesis is essential for the growth and persistence of solid tumors and their metastases (Folkman et al. (1989) Nature 339:58-61; Hori et al. (1991) Cancer Research 51:6180-84; Kim et a. (1993) Nature 362:841-844; Millauer et al. (19.96) Cancer Research 56:1615-20). To stimulate angiogenesis, tumors upregulate their production of a variety of angiogenic factors, including the fibroblast growth factors (FGF and BFGF) (Kandel et al. (1991) Cell. 66:1095-104) and vascular endothelial cell growth factor/vascular permeability factor (VEGF/VPF). However, many malignant tumors also generate inhibitors of angiogenesis, including angiostain and thrombospondin (Chen et al. (1195) Cancer Research 55:4230-33; Good et al. (1990) Proc Natl Acad Sci USA. 87:6624-28; O'Reilly et al. (1994) Cell 79:315-28). It is postulated that the angiogenic phenotype is the result of a net balance between these positive and negative regulators of neovascularization (Good et al. (1990), supra; O'Reilly et al. (1994), supra; Parangi et al. (1996) Proc Natl Acad Sci USA. 93:2002-07; Rastinejad et al. (1989) Cell 56:345-55).

Several other endogenous inhibitors of angiogenesis have been identified, although not all are associated with the presence of a tumor. These include platelet factor 4 (Gupta et al. (2000) Blood 95:147-55), interferon-alpha, interferon-inducible protein 10 (Angiolilloet al. (1996) Ann. N.Y. Acad. Sci. 795:158-67; Strieter et al. (1995) J. Biol. Chem. 270:27348-57), which is induced by interleukin-12 and/or interferon-gamma, gro-beta (Cao et al. (1995) J. Exp. Med. 182:2069-77), and the 16 kDa N-terminal fragment of prolactin (Clapp et al. (1999) Invest. Ophthalmol. Vis. Sci. 40:2498-505). The only known angiogenesis inhibitor which specifically inhibits endothelial cell proliferation is angiostatin (O'Reilly et al. (1994), supra). Angiostatin is an approximately 38 kiloDalton (kDa) specific inhibitor of endothelial cell proliferation. Angiostatin is an internal fragment of plasminogen containing at least three of the five kringles of plasminogen Angiostatin has been shown to reduce tumor weight and to inhibit metastasis in certain tumor models. (O'Reilly et al. (1994), supra). Assays used for measuring effect on angiogenesis activity include corneal pocket assay (Invest Ophtalmol. Vis. Sci. 1999, 40: 2498-505), chorioalantoic membrane angiogenesis assay (CAM) (Am. J. Pathol. 1983, 111(3):282-7), and endothelial cell proliferation and migration assays (Biochem. Biophys. Res. Comm. 2000,271:499-508).

### SUMMARY OF THE INVENTION

We have now discovered that the Ryk protein possesses a novel biological activity in regulating angiogenesis. This activity has been demonstrated via in vitro and in vivo assays for detecting molecules having activity in modulating angiogenesis. The activity was measured using variant Ryk proteins. As used herein, a “variant Ryk protein” is intended to include a Ryk protein which lacks the transmembrane portion of the protein (e.g., the extracellular domain the Ryk protein) or fragments derived therefrom, as described further below, where the variant Ryk protein exhibits activity in regulating angiogenesis.

The instant invention encompasses the use of a variant Ryk protein for regulating or modulating angiogenesis. The current invention further encompasses the use of a variant Ryk protein for the treatment of a disease or clinical condition where angiogenesis is relevant to the causation or treatment of the disease or clinical condition, including but not limited to cancer, wound healing, diabetic retinopathies, macular degeneration, and cardiovascular diseases. Further uses of the protein include treatment of clinical conditions involving angiogenesis in the reproductive system, including regulation of placental vascularization or use as an abortifacient The instant invention also encompasses pharmaceutical compositions containing a variant Ryk protein and the use of the pharmaceutical compositions for the treatment of the above-mentioned diseases or clinical conditions.

In accordance with one aspect of the present invention, there are provided novel mature polypeptides comprising the amino acid sequence shown in FIG. 1 (SEQ ID NO: 1), as well as biologically active and diagnostically or therapeutically useful fragments, analogues and derivatives thereof. In accordance with a second aspect of the present invention, there are provided novel mature polypeptides comprising the amino acid sequence shown in FIG. 2 (SEQ ID NO: 2), as well as biologically active and diagnostically or therapeutically useful fragments, analogues and derivatives thereof In accordance with a third aspect of the present invention, there are provided novel mature polypeptides comprising the amino acid sequence shown in FIG. 3 (SEQ ID NO: 3), as well as biologically active and diagnostically or therapeutically useful fragments, analogues and derivatives thereof. As an additional aspect of the present invention, there are provided antibodies to the polypeptides of the present invention, especially antibodies which bind specifically to an epitope made up of the sequences shown in FIGS. 1-3 (SEQ ID NOS: 1-3) or a sequence which shares at least a 60%, preferably at least a 70%, more preferably at least an 80%, still more preferably a 90%, or most preferably at least a 95% sequence identity over at least 20, preferably at least 30, more preferably at least 40, still more preferably at least 50, or most preferably at least 100 residues to the sequences shown in FIGS. 1-3 (SEQ ID NOS: 1-3).

In accordance with another aspect of the present invention, there are provided isolated nucleic acid molecules encoding the polypeptides of the present invention, including mRNAs, DNAs, cDNAs, genomic DNA, as well as antisense analogs thereof and biologically active and diagnostically or therapeutically useful fragments thereof

In accordance with still another aspect of the present invention, there are provided processes for producing such polypeptides by recombinant techniques through the use of recombinant vectors. As a further aspect of the present invention, there are provided recombinant prokaryotic and/or eukaryotic host cells comprising a nucleic acid sequence encoding a polypeptide of the present invention.

In accordance with a further aspect of the present invention, there is provided a process for utilizing such polypeptides, or polynucleotides encoding such polypeptides, for therapeutic purposes involving the regulation of angiogenesis, for example, the treatment of cancer, wound healing, diabetic retinopathies, macular degeneration, cardiovascular diseases, and clinical conditions involving angiogenesis in the reproductive system, including regulation of placental vascularization or use as an abortifacient.

In accordance with another aspect of the present invention, there are provided nucleic acid probes comprising nucleic acid molecules of sufficient length to specifically hybridize to a polynucleotide encoding a polypeptide of the present invention.

In accordance with yet another aspect of the present invention, there are provided diagnostic assays for detecting diseases or susceptibility to diseases related to mutations in a nucleic acid sequence of the present invention and for detecting over-expression or underexpression of the polypeptides encoded by such sequences.

In accordance with another aspect of the present invention, there is provided a process involving expression of such polypeptides, or polynucleotides encoding such polypeptides, for purposes of gene therapy. As used herein, gene therapy is defined as the process of providing for the expression of nucleic acid sequences of exogenous origin in an individual for the treatment of a disease condition within that individual.

### BRIEF DESCRIPTION OF THE FIGURES

FIG. 1 shows the amino acid sequence of the extracellular domain of the Ryk protein as described by Tamagnone et al., as well as the corresponding nucleotide sequence.

FIG. 2 shows the amino acid sequence of the extracellular domain of the Ryk protein as described by Halford et al., J. Biol. Chem. 1999; 274:7379, as well as the corresponding nucleotide sequence.

FIG. 3 shows the amino acid sequence of a portion of the extracellular domain of the Ryk protein consisting of the Wnt inhibitory factor (WIF) domain, as well as the corresponding nucleotide sequence.

FIGS. 4A and 4B show the dose dependent inhibitory effect of Ryk-Fc on HUVEC capillary-like organization in MATRIGEL matrix. Results are expressed as percentage of control, which represents the capillary-like organization of untreated HUVEC in MATRIGEL matrix.

FIG. 5 shows the dose dependent inhibitory effect of Ryk-myc-His containing supernatant on HUVEC capillary-like organization in MATRIGEL matrix. Results are expressed as percentage of control, which represents the capillary-like organization of untreated HUVEC in MATRIGEL matrix.

FIG. 6 shows the dose dependent inhibitory effect of Ryk-Fc on HUVEC capillary-like organization in MATRIGEL matrix in the presence of exogenously added bFGF, VEGF or IL-8. Results are expressed as percentage of control, which represents the capillary-like organization of untreated HUVEC in MATRIGEL matrix.

FIGS. 7A and 7B show the dose dependent inhibitory effect of WIF, WIF-Fc, NWIF-bis, WIF-his and Ryk-his on HUVEC capillary-like organization in MATRIGEL matrix in the presence of exogenously added IL-8. Results are expressed as percentage of control, which represents the capillary-like organization of untreated HUVEC in MATRIGEL matrix.

FIG. 8 shows the specificity of the Ryk-Fc fusion protein using the single chain antibody generated against the Ryk-Fc fusion protein (scFv 1b4).

FIG. 9 shows the effect of the expression of Ryk-Fc on the growth of B16 melanoma lung metastases in vivo in syngeneic mice. B16 cells were infected ex vivo with an adenovirus containing the Ryk-Fc gene and implanted in mice by tail vein injection. Results are expressed as number of lung metastases.

FIG. 10 shows the effect of the expression of Ryk-Fc on angiogenesis in vivo in the rat cornea. Hydron pellets containing the angiogenic agent, recombinant bFGF, and varying concentrations of Ryk-Fc were implanted into the rat cornea and angiogenesis was examined microscopically seven days later. Grades of angiogenesis were scored blindly by two individuals using points of reference.

FIG. 11 shows the purification of the anti-Ryk scFv antibody using Mono Q 5/5 ion-exchange chromatography. A linear gradient of 0 to 0.5 M NaCl in 10 mM Hepes buffer was used to elute the antibody from the ion-exchange column Fractions were monitored by A280 nm and conductivity value. Peaks 1 and 2 were found to be the scFv by SDS-PAGE.

FIG. 12 shows the purification anti-Ryk scFv 1B4 and endotoxin removal using Mono Q 5/5 ion-exchange chromatography. A linear gradient of 0 to 0.5 M NaCl in 10 mM Hepes buffer was used to elute the antibody from the ion-exchange column. Endotoxin was measured to 4.0 EU/ml.

FIG. 13 shows an SDS-PAGE of the anti-Ryk scFv Mono Q 5/5 purified antibody, highly purified from E. Coli. An aliquot of the Mono Q 5/5 elution (lane 1) and molecular size markers of the indicated size in kDa (lane 2) were resolved on a 4-12% bis-tris gel (Novex, Carlsbad, Calif.) and developed with coomassie stain.

FIG. 14 shows the interaction of the anti-Ryk scFv antibody with Ryk-Fe. The BIAcore 2000 instrument was used to characterize the interaction of the scFv antibody with Ryk-Fc. Data was collected using at least 6 different concentrations of Ryk-Fc, injected in duplicate. All experiments were conducted at 25° C. using parameters discussed herein.

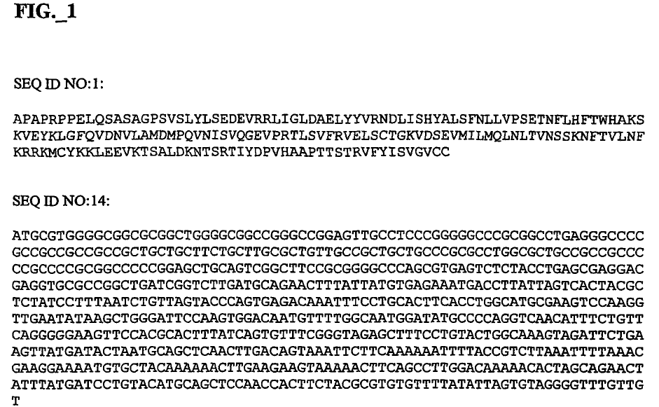

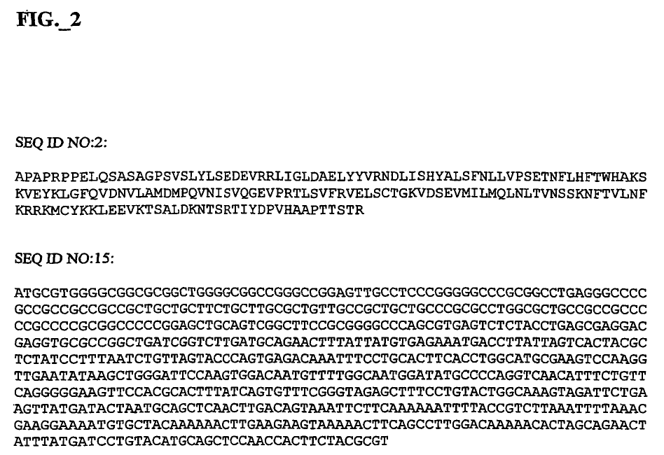

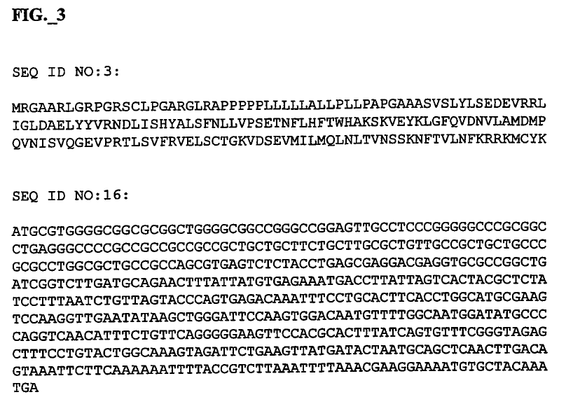

### DETAILED DESCRIPTION

The variant Ryk proteins of the present invention comprise full length, wild type Ryk proteins which lack the transmembrane portion, thereby rendering the protein soluble. Preferably, the variant Ryk proteins of the present invention comprise the extracellular domain (“ECD”) of the fill length, wild type Ryk protein, or a fragment the ECD which maintains the ability to modulate angiogenesis. Slight variations of the amino acid sequence of the Ryk protein's extracellular domain have been described in the literature by at least two different groups (See e.g., Tamagnone et al., Oncogene 1993; 8:2009 (the extracellular domain is described as having an amino acid sequence consisting of 191 amino acids (SEQ ID NO: 1) and Halford et al., J. Biol. Chem. 1999; 274:7379 (the extracellular domain is described as having the same amino acid sequence as described by Tamagnone; however, ten amino acids at the C terminus are missing making the extracellular domain 181 amino acids (SEQ ID NO: 2)); these references are incorporated herein in their entirety. For the purposes of this invention, however, any and all variants of the extracellular domain of the Ryk protein which maintain the ability to modulate angiogenesis are included. Still more preferable, the variant Ryk proteins of the present invention comprise the Wnt inhibitory factor (WIF) domain of the extracellular domain of the full length, wild type Ryk protein (SEQ ID NO: 3), or a fragment thereof which maintains the ability to modulate angiogenesis.

In light of the foregoing, the polypeptides of the present invention include those polypeptides having the deduced amino acid sequences given by SEQ ID NOS: 1-3. The polypeptides of the present invention may include additional amino acid sequences appended to the N- or C-terminal of the peptides having the deduced amino acid sequences given by SEQ ID NOS: 1-3. The polypeptides of the present invention may be recombinant polypeptides, natural polypeptides, or synthetic polypeptides, preferably recombinant polypeptides. As used herein, “protein” is synonymous with “polypeptide.”

The present invention further relates to polypeptides which share at least a 60%, preferably at least a 70%, more preferably at least an 80%, still more preferably a 90%, yet still more preferably a 95%, or most preferably at least a 98% sequence identity over at least 20, preferably at least 30, more preferably at least 40, still more preferably at least 50, yet still more preferably at least 100, or most preferably at least 150 residues with SEQ ID NOS: 1-3. (Such polypeptides may be herein referred to as “polypeptides of the present invention”.) As used herein, a “variant Ryk protein” is intended to also include polypeptides of the present invention as referred to in this paragraph. In a preferred embodiment of the invention, the polypeptide of the present invention is at least 20, preferably at least 30, more preferably at least 40, still more preferably at least 50, or most preferably at least 100 residues long. In one preferred embodiment, the peptide of the present invention is less than 600, preferably less than 550, more preferably less than 500, still more preferably less than 480 residues long. In another preferred embodiment, the peptide of the present invention is less than 250, preferably less than 185, more preferably less than 150, still more preferably less than 100 residues long, or most preferably less than 60 residues long. The invention also contemplates polypeptides which share at least a 60%, preferably at least a 70%, more preferably at least an 80%, still more preferably a 90%, or most preferably at least a 95% sequence identity over at least 20, preferably at least 30, more preferably at least 40, still more preferably at least 50, or most preferably at least 100 residues SEQ ID NOS: 1-3. Polypeptides of the present invention exhibiting such a percent identity to sequences as above-described may be assayed for biological activity as described herein by using the MATRIGEL matrix assay, as described herein, or by using other assays directed to measuring activity in regulating angiogenesis, as are known in the art. The process of measuring for biological activity is within the scope of one skilled in the art given the disclosure herein. Modulating angiogenesis includes processes resulting in either upregulation or downregulation of angiogenic processes.

Such a polypeptide as described above may be (i) one in which one or more of the amino acid residues are substituted (as compared to SEQ ID NOS: 1-3) with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethyleneglycol), or (iv) one in which additional amino acids are fused to the mature polypeptide, such as a leader or secretory sequence or a sequence which is employed for purification of the mature polypeptide or a proprotein sequence or mature protein sequence beyond the Ryk extracellular domain, or (v) one in which one or more amino acids are deleted from or inserted into the sequence of the polypeptide. Combinations of the above-described types of variations in the peptide sequence are within the scope of the invention. Such polypeptides are deemed to be within the scope of those skilled in the art from the teachings herein.

A polypeptide of the present invention may contain amino acids other than the 20 gene-encoded amino acids. The polypeptides may be modified by either natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains, and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslational natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gammacaboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

The polypeptides and polynucleotides of the present invention are preferably provided in an isolated form, and preferably are purified to homogeneity. The term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotide could be part of a vector and/or such polynucleotide or polypeptide could be part of a composition, and still be isolated in that such vector or composition is not part of its natural environment.

As known in the art “similarity” between two polypeptides is determined by comparing the amino acid sequence and its conserved amino acid substitutes of one polypeptide to the sequence of a second polypeptide. Such conservative substitutions include those described by Dayhoff, The Atlas of Protein Sequence and Structure 5 (1978) and by Argos, EMBO J. 8: 779-785 (1989). For example, amino acids belonging to one of the following groups represent conservative changes:

ala, pro, gly, gn, asn, ser, thr;

cys, ser, tyr, thr;

val, ile, lem, met, ala, phe;

lys, arg, his;

phe, tyr, trp, his; and

asp, glu.

(Note that these grouping are examples; other groupings may represent more relevant choices.)

“Similarity” or “identity” refers to sequence conservation, or “homology”, between two or more peptides or two or more nucleic acid molecules, normally expressed in terms of percentages. When a position in the compared sequences is occupied by the same base or amino acid (“residue”), then the molecules are identical at that position. When a position in two compared peptide sequences is occupied by an amino acid with similar physical properties (a conservative substitution as determined by a given scoring matrix; similarity is thus dependent on the scoring matrix chosen), then the molecules are similar at that position. The percent identity or similarity can be maximized by aligning the compared sequences alongside each other, sliding them back and forth, and conservatively introducing gaps in the sequences where necessary. The percent identity is calculated by counting the number of identical aligning residues dividing by the total length of the aligned region, including gaps in both sequences, and multiplying by 100. Identity would thus be expressed as, e.g., “60% identity over 200 amino acids,” or “57% identity over 250 amino acids.” Altematively, such sequences would be described as being “60% identical over 200 amino acids” or “57% identical over 250 amino acids,” respectively. Similarity is calculated by counting both identities and similarities in the above calculation. For example, the alignment below has 37.5% sequence identity over 56 amino acids ((21 identities/56 residues)×100% ), where 56 is the total length of the aligned region. As used herein, “percent identity” and “percent sequence identity” are interchangeable.

| | |
|---|---|
| RTPSDKPVAH--VANPQLQWLNRRANALLANGVE-RDNQLVV--EGLYLIYSQVLF | 56 resid. |
| \| \| \| \| \|\| \| \| \| \| \|\| \| \|\| \|\|\| \| \| \| | 21 ident. |
| RAPFKKSWAYLQVAKHKLSW-NK--DGIL-HGVRYQDGNLVIQFPGLYFIICQLQF | 56 resid. |
| First sequence is SEQ ID NO:11; second sequence is SEQ ID NO:12 | |

As a further example, the same alignment below has 55.4% sequence similarity over 56 amino acids ((31 similarities/56 residues)×100% ), where 56 is the total length of the aligned region. In this example, conservative substitutions are indicated by a plus sign and the total similarities is given by the sum of the identities and the conservative substitutions. (As noted above, determination of conservative substitutions is dependent on the scoring matrix chosen. The same alignment below may yield a different value for percent similarity using a different scoring matrix.)

| | |
|---|---|
| RTPSDKPVAH--VANPQLQWLNRRANALLANGVE-RDNQLVVE--GLYLTYSQVLF | 56 resid. |
| R P K A+ VA +L W N+ + +L +GV +D LV++ GLY I Q+ F | 31 simil. |
| RAPFKKSWAYLQVAKHKLSW-NK--DGIL-HGVRYQDGNLVIQFPGLYFIICQLQF | 56 resid. |
| First sequence is SEQ ID NO:11; second sequence is SEQ ID NO:12 | |

Both of the sequences in the aligned region may be contained within longer, possibly less homologous sequences. “Unrelated” or “non-homologous” sequences typically share less than 40% identity at the peptide level, preferably less than 25% identity.

The invention further encompasses polynucleotides which code for the above-described polypeptides of the present invention. These polynucleotides may be in the form of RNA or in the form of DNA, which DNA includes cDNA, genomic DNA, and synthetic DNA. The DNA may be double-stranded or single-stranded. The polynucleotides may include: only the coding sequence for the mature polypeptide; the coding sequence for the mature polypeptide and additional coding sequence such as a leader or secretory sequence or a proprotein sequence; the coding sequence for the mature polypeptide (and, optionally, additional coding sequence) and non-coding sequence, such as introns or non-coding sequence 5′ and/or 3′ of the coding sequence for the mature polypeptide. Thus, the term “polynucleotide encoding a polypeptide” encompasses a polynucleotide which includes only coding sequence for the polypeptide as well as a polynucleotide which includes additional coding and/or non-coding sequence.

The present invention further relates to variants of the herein above-described polynucleotides. The variants of the polynucleotides may be naturally occurring allelic variants of the polynucleotides or non-naturally occurring variants of the polynucleotides. As known in the art, an allelic variant is an alternate form of a polynucleotide sequence which may have a substitution, deletion, or addition of one or more nucleotides which does not substantially alter the function of the encoded polypeptides. Thus, the present invention includes polynucleotides encoding the same mature polypeptides as described in Example 1, below, as well as variants of such polynucleotides which variants include deletion variants, substitution variants, and addition or insertion-variants.

The present invention also includes polynucleotides wherein the coding sequence for the mature polypeptides may be fused to a polynucleotide sequence which aids in expression and secretion of a polypeptide from a host cell, for example, a leader sequence which functions as a secretory sequence for controlling transport of a polypeptide from the cell. The polypeptide having a leader sequence is a preprotein and may have the leader sequence cleaved by the host cell to form the mature form of the polypeptide. The polynucleotides may also encode for a proprotein which is the mature protein plus additional amino acid residues. A mature protein having a prosequence is a proprotein and is an inactive form of the protein. Once the prosequence is cleaved an active mature protein remains. For example, the polynucleotides of the present invention may code for a mature protein or for a protein having a prosequence or for a protein having both a prosequence and a presequence (leader sequence).

The polynucleotides of the present invention may also have the coding sequence fused in frame to a marker sequence which allows for purification of the polypeptide of the present invention. The marker sequence may be, for example, a hexa-histidine tag supplied by a pQE-9 vector to provide for purification of the mature polypeptide fused to the marker in the case of a bacterial host, or, for example, the marker sequence may be a hemagglutinin (HA) tag when a mammalian host, e.g. COS-7 cells, is used. The HA tag corresponds to an epitope derived from the influenza hemagglutinin protein Wilson et al., 1984, Cell 37: 767. Other tag systems are well-known in the art, including the FLAG tag. The FLAG tag is based on the FLAG marker octapeptide (N-AspTyrLysAspAspAspAspLys-C) (SEQ ID NO: 13). The FLAG sequence is hydrophilic and the last 5 amino acids (AspAspAspAspLys) (subsequence of SEQ ID NO: 13) represent the target sequence of the protease enterokinase.

The term “gene” means the segment of DNA involved in producing a polypeptide chain; it includes regions preceding and following the coding region (leader and trailer) as well as intervening sequences (introns) between individual coding segments (exons). Fragments of a gene may be used as a hybridization probe for a cDNA library to isolate the full length gene and to isolate other genes which have a high sequence similarity to the gene or similar biological activity. Probes of this type typically have at least 20 bases and preferably have at least 30 bases and may contain, for example, 50 or more bases. The probe may also be used to identify a cDNA clone corresponding to a full length transcript and a genomic clone or clones that contain the complete gene including regulatory and promotor regions, exons, and introns. An example of a screen comprises isolating the coding region of a gene by using the known DNA sequence to synthesize an oligonucleotide probe. Labeled oligonucleotides having a sequence complementary to that of the gene are used to screen a library of human cDNA, genomic DNA or mRNA to determine which members of the library the probe hybridizes to.

The present invention is directed to polynucleotides having at least a 70% identity, preferably at least 80% identity, more preferably at least a 90% identity, still more preferably at least a 95% identity, and most preferably at least 98% identity to a polynucleotide which encodes a polypeptide of the present invention, as well as fragments thereof, which fragments have at least 20 bases and preferably have at least 30 bases and more preferably have at least 50 bases, and to polypeptides encoded by such polynucleotides. A preferred embodiment is given by polynucleotides that have at least a 70% identity, preferably at least 80% identity, more preferably at least a 90% identity, still more preferably at least a 95% identity, and most preferably at least 98% identity to a polynucleotide which encodes the polypeptides having the sequences given by SEQ ID NOS: 1-3. Such polynucleotides may be contained within larger nucleotide sequences, as in embodiments of the present invention given by SEQ ID NO: 17-20. In preferred embodiments the invention is directed to polynucleotides which are less than 1500, preferably less than 1000, more preferably less than 600, still more preferably less than 200, yet still more preferably less than 100, most preferably less than 35 bases long.

The present invention also relates to vectors that include polynucleotides of the present invention as above described, host cells that are genetically engineered with vectors of the invention, and the production of polypeptides of the invention by recombinant techniques. Host cells may be genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants, or amplifying the gene for the polypeptide of the present invention. The culture conditions, such as temperature, pH and the like, are those previously used with the host cell selected for expression, and will be apparent to the ordinarily skilled artisan. The polynucleotide of the present invention may be employed for producing a polypeptide by recombinant techniques.

Thus, for example, the polynucleotide sequence may be included in any one of a variety of expression vehicles, in particular vectors or plasmids for expressing a polypeptide. Such vectors include chromosomal, non-chromosomal and synthetic DNA sequences, e.g., derivatives of SV40; bacterial plasmids; phage DNA; yeast plasmids; vectors derived from combinations of plasmids and phage DNA, viral DNA such as vaccinia, adenovirus, fowl pox virus, and pseudorabies. However, any other vector or plasmid may be used as long as they are replicable and viable in the host.

The appropriate DNA sequence may be inserted into the vector by a variety of procedures. Such procedures and others are deemed to be within the scope of those skilled in the art. The DNA sequence in the expression vector is operatively linked to an appropriate expression control sequence(s) (promoter) to direct mRNA synthesis. As representative examples of such promoters, there may be mentioned: LTR or SV40 promoter, the E. coli. lac or trp, the phage lambda PL promoter and other promoters known to control expression of genes in prokaryotic or eukaryotic cells or their viruses. The expression vector may also contain a ribosome binding site for translation initiation and a transcription terminator. The vector may also include appropriate sequences for amplifying expression. In addition, the expression vectors preferably contain a gene to provide a phenotypic trait for selection of transformed host cells such as dihydrofolate reductase or neomycin resistance for eukaryotic cell culture, or such as tetracycline or ampicillin resistance in E. coli. The vector containing the appropriate DNA sequence as herein above described, as well as an appropriate promoter or control sequence, may be employed to transform an appropriate host to permit the host to express the protein. As representative examples of appropriate hosts, there may be mentioned: bacterial cells, such as E. coli, Salmonella typhimurium, Streptomyces; fungal cells, such as yeast; insect cells, such as Drosophila S2 and Spodoptera Sf9; animal cells such as CHO, COS or Bowes melanoma; plant cells, etc. The selection of an appropriate host is deemed to be within the scope of those skilled in the art from the teachings herein.

The present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art and are commercially available. The following vectors are provided by way of example. Bacterial: pQE70, pQE60, pQE-9 (Qiagen), pBS, phagescript, psiX174, pBluescript SK, pBsKS, pNH8a, pNH16a, pNH18a, pNH46a (Stratagene), pTRC99A, pKK223-3, pKK233-3, pDR540, PRIT5 (Pharmacia). Eukaryotic: pWLneo, pSV2cat, pOG44, pXT1, pSG (Stratagene) pSVK3, pBPV, PMSG, PSVL (Pharmacia). However, any other plasmid or vector may be used as long as they are viable or can be made viable in the host. Promoter regions can be selected from any desired gene using CAT (chloramphenicol acetyl transferase) vectors or other vectors with selectable markers. Two appropriate vectors are pKK232-8 and pCM7. Particular named bacterial promoters include laci, lacZ, T3, T7, gpt, lambda PR, PL and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

The present invention also relates to host cells containing the above-described construct. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation. The constructs in host cells can be used in a conventional manner to produce the gene product encoded by the recombinant sequence. Alternatively, the polypeptides of the invention can be synthetically produced by conventional peptide synthesizers.

Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook et al. (Molecular Cloning: A Laboratory Manual, Second Edition, Cold Spring Harbor, N.Y., 1989; the disclosure of which is hereby incorporated by reference).

Transcription of a DNA encoding the polypeptides of the present invention by higher eukaryotes is increased by inserting an enhancer sequence into the vector. Enhancers are cis-acting elements of DNA, usually from about 10 to 300 bp, that act on a promoter to increase its transcription. Examples include the SV40 enhancer on the late side of the replication origin (bp 100 to 270), a cytomegalovirus early promoter enhancer, a polyoma enhancer on the late side of the replication origin, and adenovirus enhancers. Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), alpha factor, acid phosphatase, or heat shock proteins, among others. The heterologous structural sequence is assembled in appropriate phase with translation, initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Useful expression vectors for bacterial use are constructed by inserting a structural DNA sequence encoding a desired protein together with suitable translation, initiation, and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include E.coli, Bacillus subtilis, Salmonella typhimurium and various species within he genera Pseudomonas, Streptomyces, and Staphylococcus, although others may also be employed as a matter of choice. Useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM1 (Promega Biotec, Madison, Wis.) These pBR322 “backbone” sections are combined with an appropriate promoter and the structural sequence to be expressed.

After transformation of a suitable host strain and growth of the host strain to an appropriate cell density, the selected promoter may be de-repressed, if necessary, by appropriate means (e.g., temperature shift or chemical induction) and the cells may be cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents.

Various mammalian cell culture systems can also be employed to express recombinant protein. Examples of mammalian expression systems include the COS-7 lines of monkey kidney fibroblasts (82) and other cell lines capable of expressing protein from a compatible vector, for example, the C127, 3T3, CHO, HeLa and BHK cell lines. Mammalian expression vectors will generally comprise an origin of replication, a suitable promoter and enhancer, and also any necessary ribosome binding sites, polyadenylation site, splice donor and acceptor sites, transcription termination sequences, and 5′ flanking nontranscribed sequences. DNA sequences derived from the SV40 viral genome, for example, SV40 origin, early promoter, enhancer, splice, and polyadenylation sites may be used to provide the required non-transcribed genetic elements.

The polypeptide of the present invention may be recovered and purified from recombinant cell cultures by methods used heretofore, including ammonium sulfate or ethanol precipitation, acid extraction, anion or cation exchange chromatography, phosphocellulose chromatography, hydrophobic interaction chromatography, affinity chromatography, hydroxyapatite chromatography and lectin chromatography. Protein refolding steps can be used, as necessary, in completing configuration of the mature protein. Finally, high performance liquid chromatography (HPLC) can be employed for final purification steps.

The polypeptide of the present invention may be a naturally purified product, or a product of chemical synthetic-procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated with mammalian or other eukaryotic carbohydrates or may be non-glycosylated. Polypeptides of the present invention may also include an initial methionine amino acid residue.

Polypeptides of the present invention, or polynucleotides coding for polypeptides of the present invention, may be used in a process of gene therapy. Such gene therapy may be involved in the treatment of a disease or clinical condition which may include but is not limited to cancer, wound healing, diabetic retinopathies, macular degeneration, cardiovascular diseases, and clinical conditions involving angiogenesis in the reproductive system, including regulation of placental vascularization or use as an abortifacient. For example, cells may be engineered with a polynucleotide (DNA or RNA) encoding for the polypeptide ex vivo, the engineered cells are then provided to a patient to be treated with the polypeptide. Such methods are well-known in the art. For example, cells may be engineered by procedures known in the art by use of a retroviral particle containing RNA encoding for the polypeptide of the present invention.

Both in vitro and in vivo gene therapy methodologies are contemplated. Several methods for transferring potentially therapeutic genes to defined cell populations are known. See, e.g., Mulligan (1993) Science 260: 926-31. These methods include:

1) Direct gene transfer. See, e.g., Wolff et al (1990) Science 247:1465-68.

2) Liposome-mediated DNA transfer. See, e.g., Caplen at al. (1995) Nature Med. 3: 39-46; Crystal (1995) Nature Med. 1:15-17; Gao and Huang (1991) Biochem. Biophys. Res. Comm. 179:280-85.

3) Retrovirus-mediated DNA transfer. See, e.g., Kay et al. (1993) Science, 262:117-19; Anderson (1992) Science 256:808-13. Retroviruses from which the retroviral plasmid vectors hereinabove mentioned may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus. In one embodiment, the retroviral plasmid vector is derived from Moloney Murine Leukemia Virus.

4) DNA Virus-mediated DNA transfer. Such DNA viruses include adenoviruses (preferably Ad-2 or Ad-5 based vectors), herpes viruses (preferably herpes simplex virus based vectors), and parvoviruses (preferably “defective” or non-autonomous parvovirus based vectors, more preferably adeno-associated virus based vectors, most preferably AAV-2 based vectors). See, e.g., Ali et al. (1994) Gene Therapy, 1:367-84; U.S. Pat. No. 4,797,368, incorporated herein by reference, and U.S. Pat. No. 5,139,941, incorporated herein by reference. Adenoviruses have the advantage that they have a broad host range, can infect quiescent or terminally differentiated cells, such as neurons or hepatocytes, and appear essentially non-oncogenic. Adenoviruses do not appear to integrate into the host genome. Because they exist extrachromosomally, the risk of insertional mutagenesis is greatly reduced Adeno-associated viruses exhibit similar advantages as adenoviral-based vectors. However, AAVs exhibit site-specific integration on human chromosome 19.

The choice of a particular vector system for transferring the gene of interest will depend on a variety of factors. One important factor is the nature of the target cell population Although retroviral vectors have been extensively studied and used in a number of gene therapy applications, these vectors are generally unsuited for infecting non-dividing cells. In addition, retroviruses have the potential for oncogenicity. However, recent developments in the field of lentiviral vectors may circumvent some of these limitations. See Naldini et al. (1996) Science 272:263-7.

According to this embodiment, gene therapy with DNA encoding a polypeptide of the present invention is provided to a patient in need thereof, concurrent with, or immediately after diagnosis. The skilled artisan will appreciate that any suitable gene therapy vector containing DNA encoding a polypeptide of the present invention may be used in accordance with this embodiment. The techniques for constructing such a vector are known. See, e.g., Anderson (1998) Nature,392 25-30; Verma (1998) Nature, 389 239-42. Introduction of the vector to the target site may be accomplished using known techniques.

The present invention also relates to a diagnostic assay for detecting levels of polypeptides of the present invention, e.g. in various tissues, since an overexpression of the proteins compared to normal control tissue samples may detect the presence of abnormal cellular proliferation, for example, a tumor. Assays used to detect levels of protein in a sample derived from a host are well-known to those of skill in the art and include radioimmunoassays, competitive-binding assays, Western Blot analysis, ELISA assays and “sandwich” type assays. Diagnostic assays may also include the detection of polynucleotides which code for the polypeptides of the present invention.

The polypeptides of the present invention can be used as an immunogen to produce antibodies thereto. These antibodies can be, for example, polyclonal or monoclonal antibodies. The present invention also includes chimeric, single chain, and humanized antibodies, as well as Fab fragments, or the product of an Fab expression library. Various procedures known in the art may be used for the production of such antibodies and fragments.

Antibodies generated against the polypeptides of the present invention are included in the present invention. “Antibody” as used herein includes intact immunoglobulin molecules (e.g., IgG1, IgG2a, IgG2b, IgG3, IBM, IgD, IgE, IgA), as well as fragments thereof, such as Fab, F(ab′)₂, scFv, and Fv, which are capable of specific binding to an epitope of a variant Ryk protein. Preferably, antibodies that specifically bind to variant Ryk do not detect other proteins in immunochemical assays. In addition, preferable antibodies of this invention are human antibodies.

More specifically, human antibodies of the present invention specifically bind to human variant Ryk protein with a K_{d} of about 0.1 nM to about 10 μM, about 2 nM to about 1 μM, about 2 nM to about 200 nM, about 2 nM to about 150 nM, or about 50 nM to about 100 nM. More preferred human antibodies specifically bind to human variant Ryk with a K_{d} selected from the group consisting of about 2 nM, about 7 nM about 10 nM and about 11 nM. Preferred K_{d}s range from about 1 nM, about 3 nM, about 9 nM, about 13 nM, about 14 nM, to about 15 nM. The K_{d} of human antibody binding to variant Ryk can be assayed using any method known in the art including technologies such as real-time Bimolecular Interaction Analysis (BIA) (Sjolander & Urbaniczky, Anal. Chem. 63, 2338-2345, 1991, and Szabo et al., Curr. Opin. Struct. Biol. 5, 699-705, 1995). BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

Antibodies generated against the polypeptides of the present invention can be obtained in various manners. For example, antibodies by direct injection of the polypeptides into an animal or by administering the polypeptides to an animal, preferably a nonhuman. The antibody so obtained will then bind the polypeptides itself. In this manner, even a sequence encoding only a fragment of the polypeptides can be used to generate antibodies binding the whole native polypeptides. Such antibodies can then be used to isolate the polypeptide from tissue expressing that polypeptide or as a diagnostic reagent.

For preparation of monoclonal antibodies, any technique which provides antibodies produced by continuous cell line cultures can be used. See generally Antibodies: A Laboratory Manual, Harlow and Lane, eds. (1988) Cold Spring Harbor Laboratory. Examples include the hybridoma technique (Kohler and Milstein (1975) Nature 256:495-97), the trioma technique, the human B-ell hybridoma technique (Kozbor et al. (1983) Immunology Today, 4:72), and the EBV-hybridoma technique to produce human monoclonal antibodies (Cole et al. (1985) in Monoclonal Antibodies and Cancer Therapy, Alan R. Liss, Inc., pp. 77-96).

Techniques described for the production of single chain antibodies (U.S. Pat. No. 4,946,778) can be adapted to produce single chain antibodies to immunogenic polypeptide products of this invention. Also, transgenic mice may be used to express humanized antibodies to immunogenic polypeptide products of this invention. Humanized antibodies may also be produced by methods described in U.S. Pat. Nos. 5,530,101; 5,585,089; 5,693,761; and 5,693,762, incorporated herein by reference.

Human antibodies with the variant Ryk binding characteristics described above can be identified from the MorphoSys HuCAL library as follows. Human variant Ryk protein is coated on a microtiter plate and incubated with the MorphoSys HuCAL-Fab phage library (see Example 9, below). Those phage-linked Fabs not binding to variant Ryk can be washed away from the plate, leaving only phage which tightly bind to variant Ryk. The bound phage can be eluted by a change in pH and amplified by infection of E. coli hosts. This panning process can be repeated once or twice to enrich for a population of antibodies that tightly bind to variant Ryk. The Fabs from the enriched pool are then expressed, purified, and screened in an ELISA assay. The identified hits are then screened in the enzymatic assay described in Bickett et al., 1993, and Bodden et al., 1994.

Details of the screening process are described in the specific examples, below. Other selection methods for highly active specific antibodies or antibody fragments can be envisioned by those skilled in the art and used to identify human TIMP-1 antibodies.

Human antibodies with the characteristics described above also can be purified from any cell that expresses the antibodies using methods known to the skilled artisan, and which are described above relating to the purification of a protein. Moreover, human antibodies can be produced using chemical methods to synthesize its amino acid sequence, again, using techniques well known to the skilled artisan and described above relating to the purification of a protein.

### SPECIFIC EMBODIMENTS

### EXAMPLE 1

Expression of Ryk-Fc fusion protein: The cDNA corresponding to the extracellular domain of Ryk as described by Halford (SEQ ID NO: 2) was fused at its 3′ end to the cDNA encoding the Fc tag (SEQ ID NO: 4) and at its 5′ end to the cDNA encoding the signal peptide sequence (SEQ ID NO: 5). The resulting fusion gene (polynucleotide sequence given in SEQ ID NO: 16) was cloned into a vector containing an hCMV promoter to drive gene expression and an ampicillin resistance gene for selection, and the protein was expressed in hEK293E cells (Invitrogen, Carlsbad, Calif.). The Ryk-Fc fusion protein having the sequence given by SEQ ID NO: 8 (lacking the signal peptide sequence, which is removed during processing by the cells during secretion) was purified from conditioned medium by a one step purification using a Protein-A-Sepharose affinity resin. Elution of the Ryk-Fc fusion protein from the affinity resin was performed with 0.2 M glycine, pH 2.8. The eluate was immediately neutralized with saturated sodium bicarbonate to pH 7.5-8.0 and dialyzed into phosphate buffered saline with dialysis membrane (6,000 dalton cut-oft) overnight at 5° C. Analytical analysis of the purified Ryk-Fc fusion protein by SDS-PAGE and Western blot analysis with anti-Fc antibodies indicated the presence of two components, one at the expected molecular mass of the monomer (˜60 kDa) and the other at the mass predicted for the dimer (˜120 kDa).

Expression of Ryk-myc-His fusion protein: This variant Ryk protein described by SEQ ID NO: 9 was prepared in an adenoviral expression system. A cDNA corresponding to the Ryk extracellular domain as described by Tamagnone (SEQ ID NO: 1) was fused to cDNAs corresponding to a signal sequence peptide (SEQ ID NO: 5) and a myc-(6×)His sequence (SEQ ID NO: 21). The resulting cDNA, described by SEQ ID NO: 17, was cloned into the BglII-HindIII sites of a CMV shuttle. (He et al. (1998) Proc Natl Acad Sci USA. 95:2509-14.) This plasmid is known as RYK-myc-his6 pShuttleCMV. 293A cells (Quantum Biotechnologies) were grown on a 6 well plate in DMEM+10% FBS. Once cells had reached confluence, they were rinsed two times with OPTI-MEM 1 reduced serum medium (Life Technologies, Gaithersburg, Md.) and incubated with LIPOFECTAMINE transfection solution. The transfection solution was produced according to the supplier's information (Gibco BRL, Gaithersburg, Md.). Briefly, 3 μg of RYK-myc-his6-pShuttleCMV DNA was diluted in 0.25 ml OPTIMEM 1 reduced serum medium and mixed with 30 μl LIPOFECTAMINE transfection solution that was diluted in 0.25 ml OPTI-MEM 1 reduced serum medium. The mixture was added to the cells and incubated for 4.5 hours. DMEM+10% FBS was added and incubated over night. The next day the medium was changed to DMEM 10% FBS. After another 24 hours the medium was changed to OPTI-MEM 1 reduced serum medium. 293A cells which had not been transfected were also incubated in OPTI-MEM 1 reduced serum medium to serve as a control in subsequent assays. The supernatant was collected after 48 hours of incubation.

### EXAMPLE 2

One method of evaluating angiogenic mechanisms involves the use of an in vitro assay identified as the HUVEC MATRIGEL matrix assay. This assay mimics endothelial cell capillary organization and is a standard in vitro assay used to evaluate angiogenic mechanisms.

Generally, Human umbilical cord endothelial cells (HUVEC, from ATCC, Manassas, Va.) were seeded at 3×10⁴ cells per well in HUVEC complete medium. The HUVEC complete medium contained F12K medium with 2 mM L-glutamine, 100 ug/ml Heparin, 50 ug/ml endothelial cell growth supplement (ECGS), and 10% fetal bovine serum (FBS). Murine lung endothelial cells (MLuEC) were seeded at 5×10⁴ cells per well in a complete medium containing DMEM with 2 mM L-glutamine, 1% Pen/Strep, and 10% FBS. MATRIGEL basement membrane matnx (Becton Dickinson, Franklin Lakes, N.J.) was prepared using pre-cooled pipettes, tips, plates and tubes during handling of the matrix. The matrix was thawed at 4° C. overnight on ice, used to coat a 24-well plate (Costar, V W R, West Chester, Pa.) at 0.3 ml/well, and then polymerized at 37° C. for 2 hours. Test samples were added in 0.5 ml of complete medium per well, and either HUVEC or MluEC cells were added in 0.5 ml of medium per well, so the total volume of medium per well was 1.0 ml. Experiments were conducted in triplicate, with varying protein concentration (from 2.5 fM to 250 nM with log increment, or from 1:4 to 1:4000 dilution of variant Ryk protein-containing supernatant). Cells were incubated overnight at 37° C., 5% CO₂, then fixed and stained using a DIFF-QUIK staining set (VWR, West Chester, Pa.). Plates were dipped in Fixative Solution for 5 seconds, in Solution 1 for 5 seconds, and in Solution 2 for 5 seconds, then rinsed with deionized water and allowed to dry. Plates were then examined under inverted microscope, and quantitative analysis of the capillary-like structures was performed. As used herein, the term “capillary-like structures” refers to the organizational structures that result from the behavior of endothelial cells in vitro on a MATRIGEL Matrix. The term “capillary-like structures” can also include organized cells in vivo or in vitro leading up to and participating in angiogenesis which results in the cells in association with each other and forming capillaries.

The effect of a variant Ryk protein was evaluated using the in vitro HUVEC Matrigel assay. Specifically, Ryk-Fc was added at different concentrations to HUVEC grown in culture on MATRIGEL matrix. Twenty-four hours later cells were fixed and evaluated for capillary-like organization. Measurement of the capillary-like organization in each well allows a quantitative analysis of the biological effect of tested compounds.

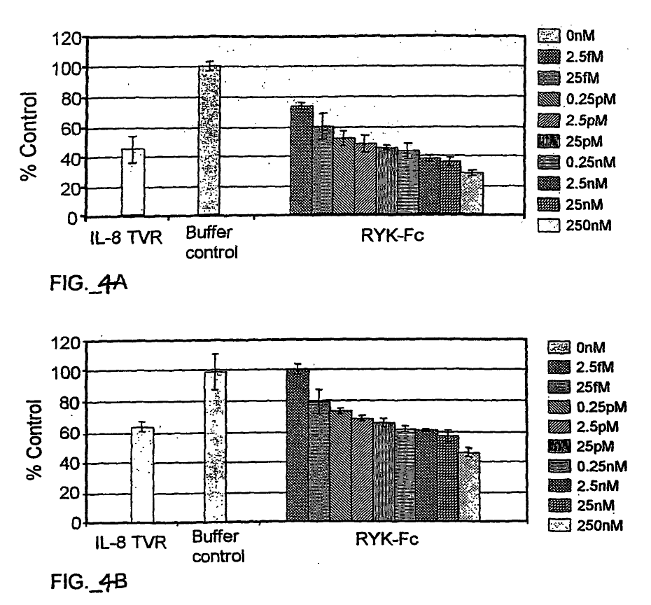

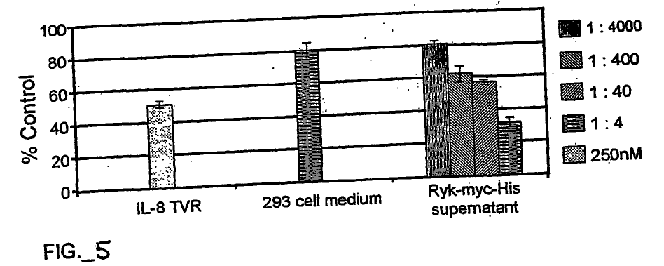

In four separate experiments, Ryk-Ec has been shown to significantly inhibit capillary-like organization in either HUVEC (FIG. 4A) or MLUEC (FIG. 4B). Results from representative experiments are presented in FIG. 4. In these experiments Ryk-Fc was prepared as described in Example 1 and was then added to the cells at various concentrations from 2.5 fM to 250 nM with log increment. The results displayed in FIGS. 4A and 4B show that Ryk-Fc inhibited capillary-like organization in a dose dependent manner. In these experiments, IL-8-TVR, an IL-8 mutein that has been shown to have an inhibitory effect in this assay, was added at a concentration of 250 nM. IL8-TVR has the two amino acids Thr-Val substituted for the two amino acids Glu-Leu, respectively, found at positions 3 and 4 of the wild-type IL-8 protein amino acid sequence. As a negative control, buffer (alone) was added to the cells at a volume equivalent to the highest volume added with Ryk-Fc, with no significant effect. In addition, endotoxin at concentrations of 0.1 Eu/mL and 1 Eu/ml was tested to determine if endotoxin could be responsible for the inhibitory effect. The concentration of endotoxin in the Ryk-Fc treated wells varied from 0.7×10⁻⁸ Eu/ml to 0.7 Eu/ml for the 2.5 fM and 250 nM Ryk-Fc concentration, respectively. The results show that endotoxin alone cannot be responsible for the inhibitory effect (data not shown).

### EXAMPLE 3

The effect of another variant Ryk protein was evaluated in vitro using the HUVEC MATRIGEL matrix assay. Ryk-myc-His was made in 293 cells transfected with a plasmid construct containing the Ryk-myc-His gene as described above in Example 1. In an experimental setup similar to that of Example 2, the Ryk-myc-His fusion protein-containing supernatant was added at various dilutions to HUVEC grown in culture on MATRIGEL matrix. Twenty-four hours later cells were fixed and evaluated for capillary-like organization.

Ryk-myc-His has been shown to significantly inhibit HUVEC capillary-like organization. Results from a representative experiment are presented in FIG. 5. In this experiment, supernatants obtained from cell cultures expressing the Ryk-myc-His fusion were added to the cells at various dilution factors from 1:4000 to 1:4 with log increment. The results displayed in FIG. 5 show that Ryk-myc-His containing supernatant inhibited capillary-like organization in a dose dependent manner. In this experiment, supernatant obtained from cells which did not express any variant Ryk protein (293 cell medium) was added at a dilution factor of 1:4 as a negative control and was shown to have no effect on HUVEC capillary-like organization.

### EXAMPLE 4

The effect of Ryk-Fc fusion protein in combination with known angiogenic factors including bFGF, VEGF and IL-8 was tested at different concentrations in HUVEC grown in culture on MATRIGEL matrix. Twenty-four hours later cells were fixed and evaluated for capillary-like organization.

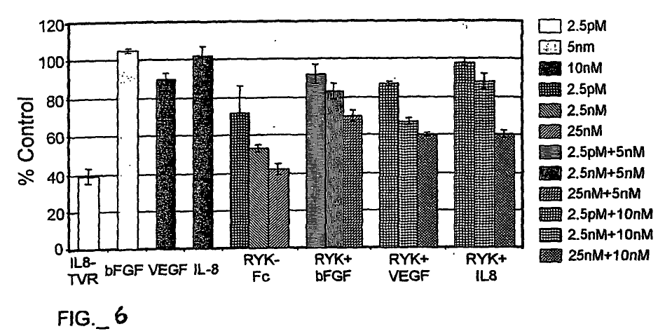

In three separate experiments, Ryk-Fc has been shown to significantly inhibit capillary-like organization regardless of the presence of bFGF, VEGF or IL-8. Results from a representative experiment are presented in FIG. 6. In this experiment Ryk-Fc was added to the cells at 2.5 pM, 2.5 nM and 25 nM in combination with either bFGF (at 5 nM, VEGF (10 nM), or IL-8 (10 nM). The results displayed in FIG. 6 show that Ryk-Fc inhibited capillary-like organization in a dose dependent manner alone or in combination with bFGF, VEGF or IL-8. In this experiment IL-8-TVR was added at a concentration of 250 nM as a positive control. In addition, bFGF, VEGF and IL-8 were tested alone and shown to have no effect on HUVEC capillary-like organization. This result suggests that angiogenic stimuli necessary for HUVEC capillary-like organization are already present either in the MATRIGEL Matrix or in the medium.

### EXAMPLE 5

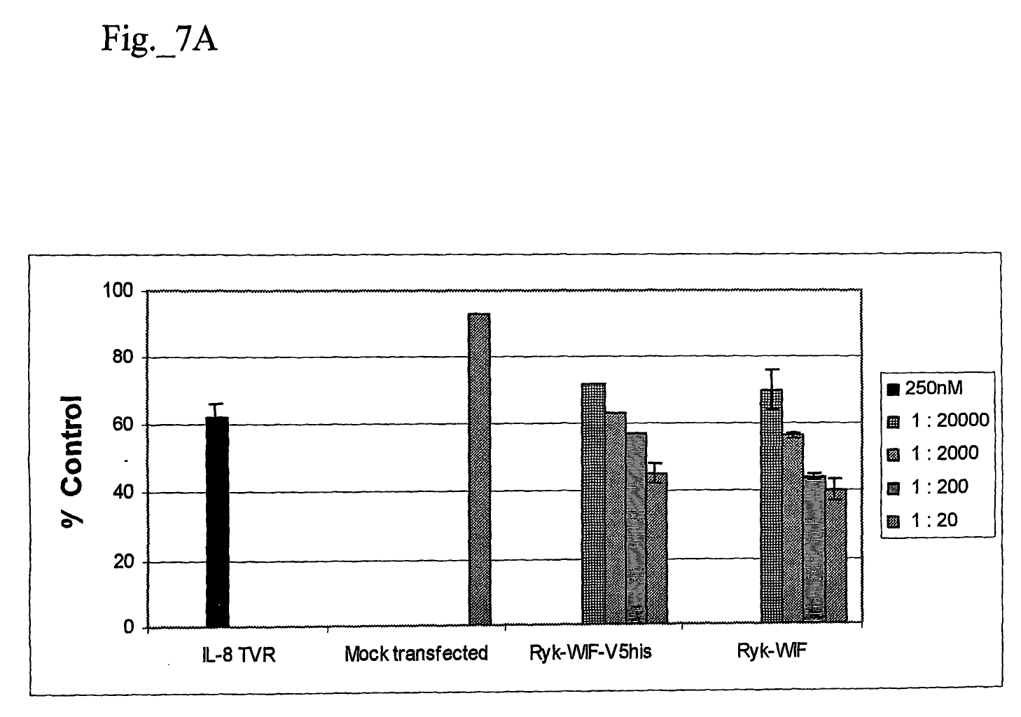

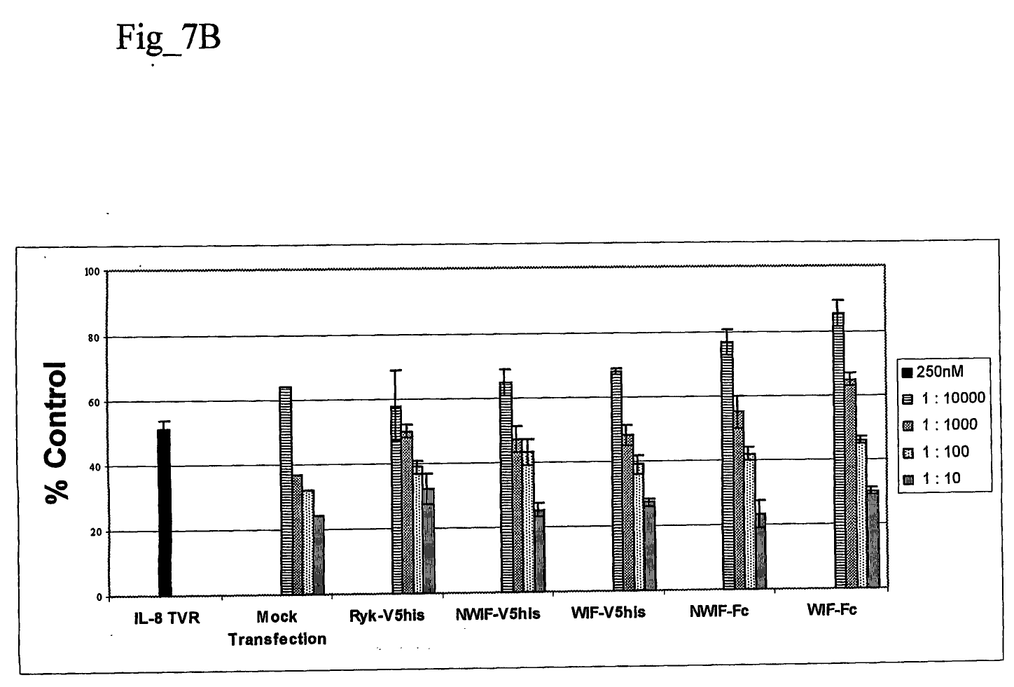

The effect of still other variant Ryk proteins were evaluated in vitro using the HUVEC MATRIGEL matrix assay. Ryk-his, WIF, WIF-Fc, N-WIF-Fc, WIF-his and N-WIF-his were tested in an experimental setup similar to that of Example 2. The variant Ryk protein-containing supernatants were added at various dilutions to HUVEC grown in culture on MATRIGEL matrix. Twenty-four hours later cells were fixed and evaluated for capillary-like organization. Results from a representative experiment are presented in FIGS. 7A and 7B. In this experiment, supernatant obtained from cells which did not express any variant Ryk protein (293 cell medium), was tested as well as IL8, as controls.

### EXAMPLE 6

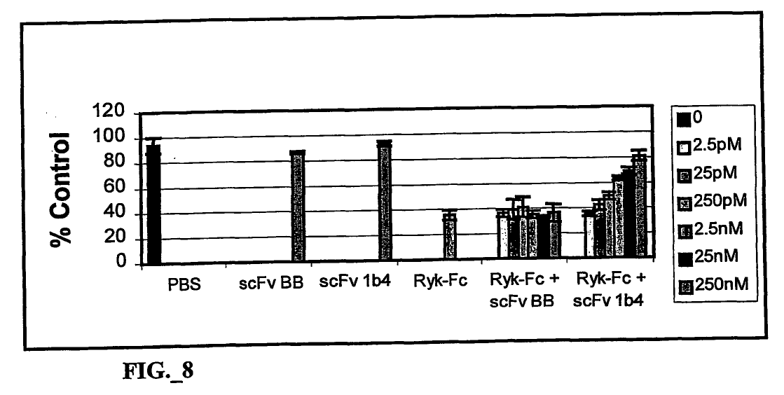

The specificity of the Ryk-Fc fusion protein was also evaluated in vitro using the HUVEC MATRIGEL matrix assay. Briefly, a single chain antibody was generated against the Ryk-Fc fusion protein (scFv1b4). Details concerning the generation of this antibody are described in the examples below. In this experiment, however, HUVEC were cultured with 2.5 nM Ryk-Fc fusion protein and increasing concentrations of scFv1b4. Twenty-four hours later cells were fixed and evaluated for capillary-like organization. Results from a representative experiment are presented in FIG. 8. In this experiment, scFvBB served as a negative control. ScFvBB is a single chain antibody that was generated against human placental bikunin using methods described in examples below.

### EXAMPLE 7

The effect of Ryk-Fc expression was evaluated in vivo in the B16 melanoma tumor model. Generally speaking, in this model, Murine B16.F10 melanoma cells (CRL-6475, American Type Culture Collection, Manassas, Va.) were infected with non-replicative adenoviral particles containing the gene encoding for RYK-Fc (Ad-RYK-Fc), Interleukin-2 (Ad-IL2) or VEGF (Ad-VEGF). Twenty-four hours after infection, cells were trypsinized, washed and resuspended in PBS. An aliquot of cells (2×10⁵ cells in a volume of 200 μl) was then injected intravenously into the tail vein of C57BL/6 female mice (Charles River Laboratories, Wilmington, Mass.) The animals were sacrificed 14 days post injection, and the lungs were collected, fixed and analyzed for weight and metasases count.

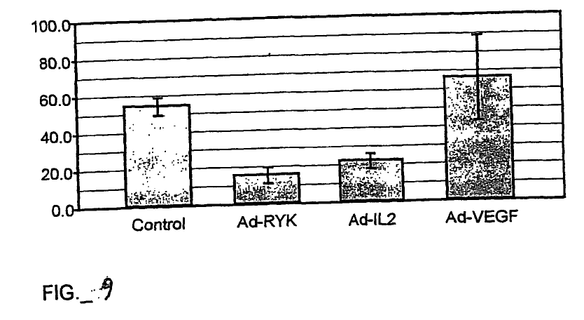

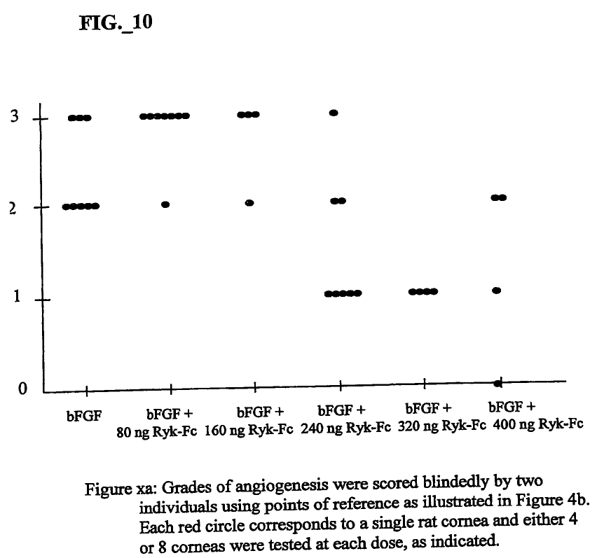

Specifically, in this example, B16-F10 cells were infected ex vivo with non-replicative adenoviral particles containing the Ryk-Fc gene (ad-Ryk-Fc) for 24 hours and then implanted in C57BL/6 mice (Charles River Laboratories, Wilmington, Mass.) intravenously through the tail vein. We have previously shown with various adenoviral constructs that ex vivo infection of B16 cells led to in vivo expression of the protein of interest for at least 6 days. The B16-F10 cells have also previously been shown to colonize the lungs and form metastases when injected intravenously. At 14 days post cell implantation, mice were sacrificed and the lungs were collected. In this model, tumor burden is evaluated by counting lung metastases. In this experiment, the controls included mice receiving untreated B16-F10 cells or B16-F10 cells that had been infected ex vivo with non-replicative adenoviral particles containing the IL-2 gene (ad-IL-2) or the VEGF gene (ad-VEGF). In previous experiments the expression of IL-2 was shown to have an inhibitory effect on lung metastases whereas the expression of VEGF was shown to have a stimulatory effect. The results presented in FIG. 9 show that B16-F10 cells infected with ad-Ryk-Fc gave rise to lower metastasis number Char non-infected B16-F10 cells. In this experiment, the expression of IL-2 also inhibited tumor growth whereas the expression of VEGF stimulated tumor growth.

### EXAMPLE 8

The effect of variant Ryk protein was tested in vivo in the rat cornea model. In this model, recombinant bFGF is dissolved in a hydron pellet that allows for sustained release of bFGF. Hydron pellets are comprised of 1% Sucrose Octasulfate Aluminum Complex, 12% Hydron Polymer Type NCC in 95% alcohol. All ingredients are mixed at room temperature overnight. Then, bFGF is added to the suspension under sterile conditions. Test materials with possible anti-angiogenic activity, such as the Ryk-Fc fusion protein, are included in the hydron pellet with bFGF.

To generate the pellets, 5 microliters of the suspension are dispensed onto sterile plastic surface. Each pellet would normally contain 400 ng of bFGF and varying amounts of test materials. In this example, each pellet contained 400 ng of bFGF and Ryk-Fc fusion protein was included at doses ranging from 80 ng to 400 ng. Pellets will form at room temperate within 2 hours.

The implantation of pellets is performed on rats that are under anesthesia. Under aseptic conditions, a micropocket (2 mm×2 mm) is produced surgically near the center of the cornea with a pliable iris spatula. The hydron pellets are pre-wetted with saline, implanted and dissolved in rat corneas. Rats are sacrificed 7 days later. Visualization of cornea angiogenesis is enhanced by heart perfusion with Indian Ink. Rat corneas are isolated, fixed in formalin overnight, and mounted onto slides. The level of agogenesis is examined microscopically. The extent of angiogenesis is scored double-blindly: 0 for no angiogenesis; 1 for modest angiogenesis; 2 for significant angiogenesis and 3 for extensive angiogenesis.

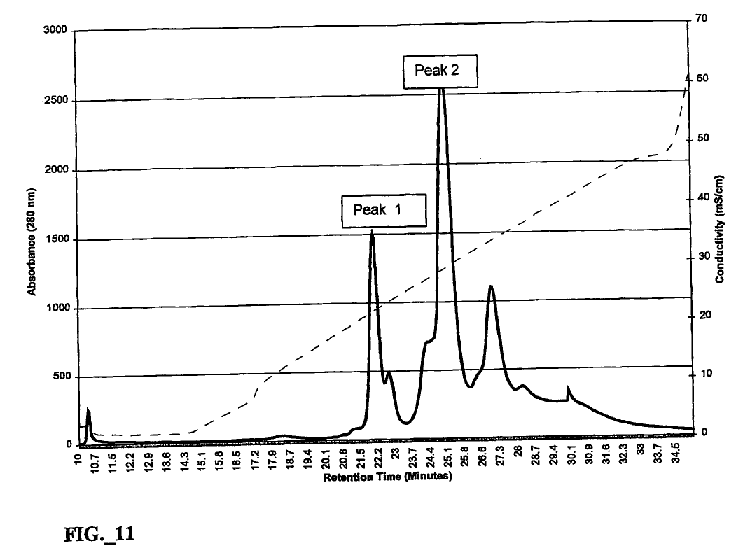

The results suggest that Ryk-Fc exhibits a dose-dependent inhibition of bFGF-induced angiogenesis in this model (FIG. 10) with a clear reduction in angiogenesis apparent at or above 240 ng Ryk-Fc.

### EXAMPLE 9

Human antibodies against variant Ryk protein were generated as follows:

Fully Synthetic Human Combinatorial Antibody Libraries (HuCAL) Based on Modular Consensus Frameworks and CDRs Randomized with Trinucleotides (Achim Knappik, Liming Ge, Annemarie Honegger, Peter Pack, Melanie Fischer, Günter Wellnhofer, Adolf Hoess, Joachim Wölle, Andreas Plückthun, Bernhard Virnekäs Journal of Molecular Biology, Vol. 296, No. 1, February 2000, pp. 57-86) is a reference that details HuCAL libraries and is incorporated by reference herein in its entirety.

HuCAL Phage Selection:

Three wells of a Maxisorp microtiter plate (F96 Maxisorp Nunc-Immuno Plate) were coated with 200 μl Ryk-Fc at a concentration of 50 μg/in PBS incubated at 64 at 4° C. The antigen solution was removed and the wells washed 2× with 400 μl PBS, then blocked with 400 μl 5% MPBS (PBS containing 5% skim milk powder, low fat, <1%) for 2 h at RT on a microtiter plate shaker. During this time, 100 μL of the phage preparation (10¹¹-10¹² tu) was mixed with 100 μl 5% MPBS-0.1% Tween 20 containing 312.5 μg/ml ILAR-Fc as blocldng protein, and incubated for 2 h at RT shaking gently. After blocking, the coated wells were washed 2× with 400 μl PBS. Pre-blocked phage mix (200 μL) was transferred into each coated well, the plate sealed, and incubated for 30 min at RT on the microtiter plate shaker followed by another 30 min standing at RT. The phage solution was removed and the wells were washed as follows:

1st round of selection: 3× 400 μl PBST (PBS containing 0.05% Tween 20) quick—2× 400 μl PBST for 5 min on a shaker at RT; 3× 400 μl PBS quick-2× 400 μl PBS for 5 min on a shaker at RT.

2nd round of selection: 1× 400 μl PBST quick—4× 400 μl PBST for 5 min on a shaker at RT; 1× 400 μl PBS quick—4× 400 μl PBS for 5 min on a shaker at RT.

Bound phage were eluted by incubation for 10 min with 200 μl 100 mM triethylamine. The eluate was rapidly neutralized by transferring to a tube containing 100 μl 1M Tris-HCl pH 7.0. The remaining phage in the selection well were rescued by addition of 200 μl of E. coli TG1 cells at OD₆₀₀ₘₘ of 0.8. At the same time 4.5 ml of an E. coli TG1 culture with an OD₆₀₀ₘₘ of 0.8 were added to the neutralized phage eluate and both this tube and the microtiter plate were incubated for 45 min at 37° C. without shaking. The two samples of infected E. coli TG1 were combined and centrifuged for 2 min at 5000 g. The supernatant was removed, and the pellet was resuspended and plated on a 150 mm LB/Cm/Glu (2×TY medium, 34 μg/ml chloramphenicol, 1% glucose) agar plate, which was incubated overnight at 30° C.

Rescue of Selected Phage:

The bacteria were scraped from the agar plate with 2 ml 2×TY/Cm/Glu containing 15% glycerol. Ten ml 2×TY/Cm/Glu medium (in 50 ml plastic tubes) were inoculated with 86 μl bacteria and incubated for 45 min at 37° C. in a shaking at 250 rpm. Five mL were transferred to culture tube, 50 μl helper phage were added, and the tube was incubated in a water bath for 30 min at 37° C. without shaking followed by 30 min at 37° C. in a shaker at 250 rpm. The bacteria were harvested by centrifugation at 4500 g for 5 min at 4° C. The pellet was resuspended in 25 ml 2×TY/Cm/50 mg/mL Kan/0.1 mM IPTG-without glucose, then incubated at 30° C., shaking at 250 rpm overnight.

Preparation and Subcloning of Selected Phage:

The bacteria were pelleted by centrifugation, and the phage were recovered from the supernatant by precipitation with 5 ml PEG/NaCl. The phage pellet was resuspended in 1 ml sterile PBS, cleared by centrifugation, filtered through a 0.8 μm syringe filter and stored at 4° C. The second round of selection was carried out as described above, using this phage preparation instead of the HuCAL library primary phage preparation.

After the second round of selection, the bacteria were scraped from two agar plates with 2 mL 2×TY/Cm/Glu containing 15% glycerol. 500 microliters of scraping from each plate was used directly for a miniprep. DNA was prepared by a standard miniprep method and digested with EcoRI and XbaI, The scFv band obtained from the pool of selected of HuCAL TAG-CAL clones was ligated by standard methods into the expression vector pMx7_FH. The ligation reaction was transformed into E. coli JM83 and plated on LB/Cm/Glu agar plates.

Micro-Expression of scFv:

Single colonies from the transformation were picked and innoculated both into a grid pattern on a 150 mm LB/Cm/1% Glu agar plate and into a 96-well microtiter plates containing 100 μl 2×TY/Cm/1% Glu medium. The plate was sealed with gas-permeable tape and incubated O/N at 37° C. with shaking For the micro-expression, approximately 5 μl per well of the master plate were transferred to the corresponding well of a new culture plate containing 100 μl 2×TY/Cm/0.1% Glu. The culture plate was incubated at 37° C. with shaking for 4 hours, then induced by addition of 100 μl/well of 2×TY/Cm containing 1 mM IPTG. The plate was incubated for an additional 4 h at 30° C. with shaking. The plate was centrifuged to pellet the bacteria, the supernatants were removed, and 125/well μl ice-cold BBS (100 mM boric acid, 150 mM NaCl, 2 mM EDTA, pH 8.0) was added. The plate was sealed and incubated overnight at 4° C. with shaking. The bacteria were pelleted by centrifugation, and 50 μL of the supernatants were used in the following scFv ELISA.

scFv ELISA:

A Maxisorp microtiter plate was coated with 100 μl Ryk-Fc in PBS (5 μg/ml) per well overnight at 4° C. The antigen solution was removed and the plate blocked for 2 h with 400 μl/well of 5% MPBS shaking on a microtiter plate shaker at RT. The plates were rinsed once with 400 μl per well TBST (50 mM Tris HCl, pH 7.4, 15 mM NaCl, 0.5% Tween-20), and 50 μl 5% MTBST (TBST containing 5% non-fat milk) were distributed to each well. The supernatants from the micro-expression plate above (50 μl/well) were transferred to the corresponding wells of the to the antigen-coated ELISA plate, which was incubated for 1.5 h at RT on a microtiter plate shaker. The plates were washed 5× quickly with TBST+1 mM CaCl₂. A mixture of monoclonal mouse anti-FLAG-tag antibodies M1 (Sigma F-3040) and M2 (Sigma F-3165) each diluted 1:10000 in TBST+1 mM CaCl₂ was added to the plate (100 μL/well), which was then incubated for 1 h at RT on a microtiter plate shaker. The plate was washed 5× quicldy with TBST+1 mM CaCl₂, and 100 μl anti-mouse IgG-HRP conjugate (Sigma A-6782) diluted 1:10 000 in TBST+1 mM CaCl₂ was added to each well, followed by incubation for 1 h at RT on a microtiter plate shaker. The plate was washed 5× quickly with TBST+1 mM CaCl₂, and 100 μl/well Peroxidase substrate BM blue soluble (Roche) was added followed by incubation for 30 min at RT. The ELISA signals were read at 370 nm.

Specificity ELISA:

Maxisorp microtiter plates were coated as described above with the following antigens. IL4R-Fc (5 μg/mL) The ELISA was carried out as described above.

Sequencing of ELISA Positive scFv:

ELISA-positive clones were innoculated from the grid agar plate into 1.2 mL 2XYT/Cm/1% Glu in a deep-well microtiter plate, and incubated 24 h at 37° C. with shaking. DNA was prepared using the QiaRobot, and sequenced using the primers HuCAL for (TACCGTTGCTCTTCACCCC) and HuCAL rev (TTTTTCACTTCACAGGTC). The DNA sequences were compared by standard alignment methods to identify unique scFv sequences, and to determine the framework sequences of each unique clone.

Large Scale Expression and Purification of scFv with Ni Affinity Chromatography:

E. coli JM83 containing the HuCAL scFv was plated on a LB/Cm/Glu agar plate, and incubated overnight at 37° C. A 10 ml culture in 2×TY/Cm/1% Glu medium was innoculated from a single colony and incubated overnight at 30° C. in a shaker. A baffled flask containing 500 mL 2×TY/Cm/0.1% Glu medium was innoculated with 2.5 mL of the fresh overnight culture, and incubated 6 h at 30° C. in a shaker at 180 OD₆₀₀ₘₘ=0.5. The bacterial culture was cooled on ice to reach RT and IPTG was added to a final concentration of 0.5 mM. The scFv was expressed at 22° C. overnight, shaking at 180 rpm, to an OD₆₀₀ₘₘ of 0.5. The bacteria were harvested by centrifugation at 5 000 g for 20 min at 4° C. The pellet was resuspended in 100 ml pre-cooled running buffer (100 mM Tris, 1 mM EDTA pH 8.0) and disrupted using a MicroFluidics cell disruptor. Bacterial debris was removed by centrifugation at 20 000 g for 30 min at 4° C. The supernatant was filtered through a 0.2 μm filter (low protein binding) and applied to a Poros metal-chelator column from Perseptive Biosystems. After washing with 10 column volumes of running buffer, the scFv was eluted with 10 column volumes of elution buffer (running buffer+250 mM imidazole). The eluted fractions were dialyzed into PBS by standard methods, aliquotted and stored at −20° C.

### EXAMPLE 10

Expression of 1B4, Anti-Ryk Single Chain scFv Antibody (Seg IDs 20 and 21)

An overnight culture of Clone 1B4 from Morphosys antibody team (Marina Roell) was grown in 2-YT media with 1% glucose, and 34 ug/ml chloramphenicol at 30° C. Ten mls of the overnight culture was used to inoculate a 1 L culture in 2-YT media with 0.1% glucose and 34 ug/ml chloramphenicol. The culture was grown at 30° C. until an absorbance at 600 nm of 0.5 was obtained. The temperature was reduced to 25° C. and expression of the 1B4 antibody induced by the addition of 500 μl of 1 M IPTG. The cells were harvested after an overnight induction.

Purification of 1B4, Anti-Ryk Single Chain scFv Antibody

The cells were pelleted by centrifugation (2660×g) and the pellet was resuspended in 20 mM sodium phosphate, pH 7.4, 0.5 M NaCl, 10 mM imidazole containing 1 ml of a protease inhibitor cocktail (Sigma) (Buffer A) and lysed using a microfluidizer (Microfluidics, Inc) at a pressure of 15,000 psi. The supernatant was centrifuged at 10,640×g, filtered and applied to a 1 ml Poros MC (Perseptive Biosystems) metal-chelator column that had been charged with Ni+ as per manufacturer's instruction. After loading, the column was washed with buffer A, and protein was eluted with a step gradient of 30, 60, 90 and 250 mM imidazole in buffer A. The 1B4 antibody peak was dialyzed in buffer containing 10 mM Hepes, pH 7.5, and 10 mM NaCl (Buffer B) for 4 hrs at 4° C., filtered, and injected onto a Mono Q 5/5 ion-exchange column that was equilibrated in Buffer B using a flow rate of 1 ml/min. After washing with Buffer B to remove unbound material, protein was eluted using a 0 to 0.5 M NaCl gradient in Buffer B. The peak containing the anti-Ryk 1B4 antibody was collected, tested for endotoxin using a QCL-1000 test lit under manufacturer suggestions (Biowhittaker, Walkersville, Md.). Because the endotoxin level in this peak was significant (1600 EU/ml), the Mono Q column was cleansed from endotoxin contamination and antibody was reapplied by first diluting the peak 1:5 with Buffer B and re-injected. The resulted peak was reassayed for endotoxin.

BIACORE Methodology—K_{d} Values for Human Variant Ryk Antibodies which Bind to Human Variant Ryk Protein

Experiments were performed using a BIACORE 2000 (Pharmacia) at 25° C. Running buffer was BIACORE HBS-EP (10 mM HEPES, 150 mM NaCl, 3 mM EDTA, 0.005% Surfactant P20, pH 7.4). The anti-Ryk scFv 1B4 antibody was amine coupled to a CM5 sensor chip by first activating the surface with a 1:1 mixture of 1-ethyl-3-(3-dimethylaminopropyl)carbodiimide hydrochloride (FDC) (1M) and N-hydroxysuccinimide (0.25M) at 5 μl/min. The antibody was diluted to 1.8 μg/ml in 10 mM sodium acetate, pH 4.5, and injected until 250 response units RU) were obtained. Following attachment, the remaining active sites on the sensor surface were blocked with 35 μl of 1 M ethanolamine, pH 8.5, at 5 μl/min. Various concentrations of Ryk-Fc (30 to 242 nM) in running buffer were injected over the surface (60 μl injection/ 30 μl/min flow rate) to follow Ryk-Fc-antibody association. Ryk-Fc-antibody was followed immediately after each association reaction by a two minute injection of running buffer. BIAcore chip surface regeneration was accomplished by injecting 50 μl of 10 mM glycine pH 2.5 at a flow rate of 100 μl/min The association (kon) and dissociation (koff) rate constants were determined by a global fit of the data using BIAeval version 3.1 with the model A+B=AB. KD values were calculated from the ratio of koff to kon.

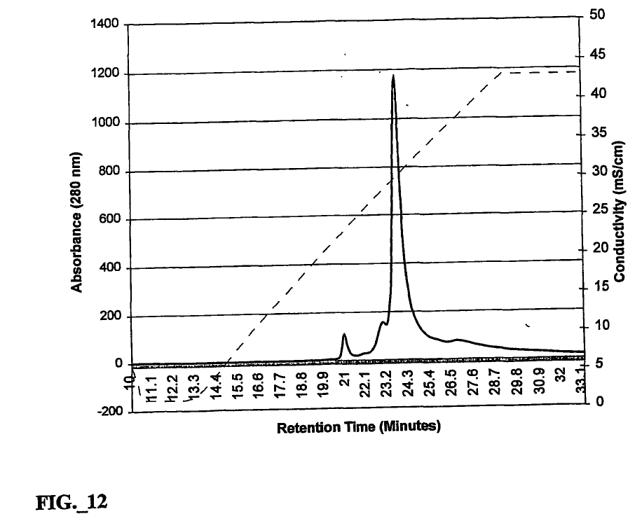

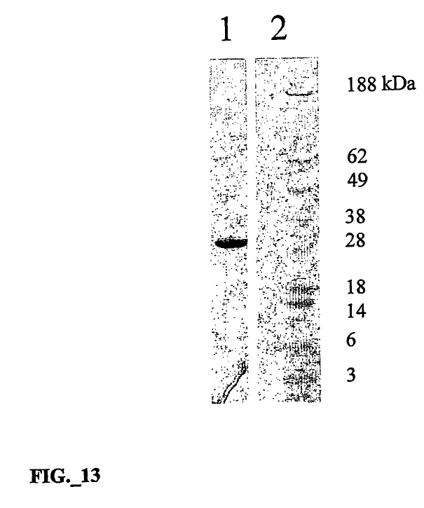

The anti-Ryk scFv 1B4 antibody was purified from E. coli lysates by sequential chelating and ion-exchange chromatography. The majority of the antibody eluted from the Poros MC chelating column at 250 mM immidizole. The anti-Ryk scFv 1B4 antibody also eluted in the earlier washes but these peaks were not combined with the high imidazole elution. A total of 11.7 A280 nm units was detected in the final pool. The fist Mono Q column elution contained multiple peaks, the first 2 of which were judged to be the scFv 1B4 antibody (FIG. 11). Because the endotoxin levels in the peaks were significant (1600 EU/ml), a subsequent Mono-Q column was run after extensive washing to remove endotoxin from the AKTA FPLC system and ion-exchange column. Peak 2 was reapplied to the Mono-Q column (FIG. 12) and the final endotoxin yield was measured to be 4.0 EU/ml. The final anti-Ryk scFv 1B4 preparation was judged to be highly pure as judged by SDS-PAGE (FIG. 13).

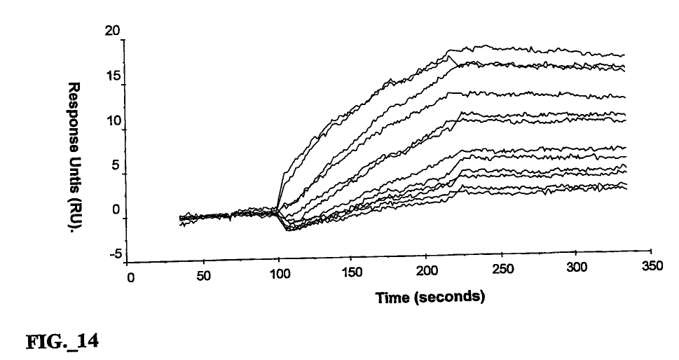

The BIAcore 2000 instrument is a powerful tool in the analysis of protein-protein interactions. Using appropriate coupling conditions, accurate association and dissociation constants for the binding of antibody to antigen can be determined. FIG. 14 shows the sensorgrams generated for the binding of various concentrations of Ryk-Fc to a fixed concentration of immobilized anti-Ryk scFv 1B4 antibody. The kinetic constants calculated from these plots are listed in Table 1 below. A KD value of 13.3 nM was calculated for this interaction, demonstrating a high affinity between the two binding partners.

TABLE 1
Kinetic binding properties of the anti-Ryk scFv antibody with Ryk-Fc

| ka (1/Ms) | Kd (sec⁻¹) | KD (M) |
|---|---|---|
| 5.0 × 10⁻⁴ | 6.7 × 10⁻⁴ | 13.3 × 10⁻⁹ |

### Conclusion

There have been no reports of Ryk involvement in angiogenesis. Though Ryk overexpression has been demonstrated in ovarian cancer its role in cancer has not been determined. We have now found that novel variant Ryk proteins quite surprisingly exhibit activity in modulating angiogenesis.

We have now discovered that the wild type Ryk protein or other variant Ryk proteins as described herein may be relevant as therapeutic agents to any disease where angiogenesis is involved, including but not limited to cancer, wound healing, diabetic retinopathies, macular degeneration, and cardiovascular diseases. The variant Ryk proteins described herein may further be used in the treatment of clinical conditions involving angiogenesis in the reproductive system, including regulation of placental vascularization, regulation of pregnancy, or use as an abortifacient. In addition to their potential therapeutic use, the polypeptides of the present invention may find use in diagnostic applications, as may the polynucleotides which code for the polypeptides of the present invention, and as may antibodies to the polypeptides of the present invention.

The above examples are intended to illustrate the invention and it is thought variations will occur to those skilled in the art. Accordingly, it is intended that the scope of the invention should be limited only by the claims below.

What is claimed is:

1. A method of modulating angiogenesis at a site, the method comprising causing an effective amount of a composition comprising a variant Ryk protein to be supplied to the site.

2. The method of claim 1 wherein the variant Ryk protein has an amino acid sequence identical to SEQ ID NO: 2.

3. The method of claim 1 wherein the variant Ryk protein has an amino acid sequence which is at least 60% identical over at least 40 residues to SEQ ID NO: 2.

4. The method of claim 1 wherein the variant Ryk protein has an amino acid sequence which is at least 70% identical over at least 30 residues to SEQ ID NO: 2.

5. A method of modulating angiogenesis at a site, the method comprising causing an effective amount of a composition comprising a variant Ryk protein to be supplied to the site, wherein the variant Ryk protein has an amino acid sequence selected from the group consisting essentially of SEQ ID NO: 1, SEQ ID NO: 2 and SEQ ID NO: 3.

6. A method of modulating the formation of cells into capillary-like structures comprising contacting the cells with a biologically effective amount of a composition comprising a variant Ryk protein, wherein the variant Ryk protein has an amino acid sequence selected from the group consisting essentially of SEQ ID NO: 1, SEQ ID NO: 2 and SEQ ID NO: 3.

7. The method of claim 6 wherein the cells are endothelial cells of human origin.

8. A protein characterized by having a deduced amino acid sequence which is at least 60% identical over 40 residues to an amino acid sequence selected from the group consisting essentially of SEQ ID NO: 1, SEQ ID NO: 2 and SEQ ID NO: 3.

9. The protein according to claim 8, wherein the deduced amino acid sequence is at least 80% identical over 50 residues to an amino acid sequence selected from the group consisting essentially of SEQ ID NO: 1, SEQ ID NO: 2 and SEQ ID NO: 3.

10. A pharmaceutical composition for modulating angiogenesis comprising a variant Ryk protein, wherein the variant Ryk protein has an amino acid sequence selected from the group consisting essentially of SEQ ID NO: 1, SEQ ID NO: 2 and SEQ ID NO: 3 and a pharmaceutically acceptable carrier.

11. A pharmaceutical composition for modulating angiogenesis comprising a variant Ryk protein, wherein the variant Ryk protein is characterized by having a deduced amino acid sequence which is at least 60% identical over 40 residues to an amino acid sequence selected from the group consisting essentially of SEQ ID NO: 1, SEQ ID NO: 2 and SEQ ID NO: 3, and a pharmaceutically acceptable carrier.

12. The method of claim 6, wherein the site is within a human patient and the protein is supplied to the site via a pharmaceutical composition according to claim 10.

13. The method of claim 12, wherein the site is within a human patient and the protein is supplied to the site via a process of gene therapy.

14. A method for preventing, treating, or ameliorating a medical condition in an individual, the method comprising providing to an individual a source of an effective amount of a variant Ryk protein, wherein the variant Ryk protein has an amino acid sequence selected from the group consisting essentially of SEQ ID NO: 1, SEQ ID NO: 2 and SEQ ID NO: 3.

15. The method of claim 14, wherein the protein is supplied to the individual by providing to the individual a source of a polynucleotide encoding the protein and expressing the protein in vivo.

16. The method of claim 14, wherein the medical condition is selected from the group consisting of cancer, metastasis, diabetic retinopathy, macular degeneration, cardiovascular disease, a wound, pregnancy, and a clinical condition involving angiogenesis in the reproductive system, including regulation of placental vascularization.

17. A polynucleotide selected from the group consisting of (a) a polynucleotide coding for a variant Ryk protein, wherein the variant Ryk protein has an amino acid sequence selected from the group consisting essentially of SEQ ID NO: 1, SEQ ID NO: 2 and SEQ ID NO 3; and (b) a polynucleotide complementary to (a).

18. The polynucleotide according to claim 17, wherein the polynucleotide is operably linked within an expression vector to a promoter.

19. The polynucleotide according to claim 17, wherein the polynucleotide is between 35 and 1500 bases long.

20. A method for producing a variant Ryk protein, wherein the variant Ryk protein has an amino acid sequence selected from the group consisting essentially of SEQ ID NO: 1, SEQ ID NO: 2 and SEQ ID NO: 3, comprising the steps of:
(a) introducing an expression vector capable of expressing the variant Ryk protein into a cell capable of expressing the variant Ryk protein,
(b) growing cells resulting from step (a) under conditions sufficient to allow the cells to express the variant Ryk protein, and
(c) recovering the variant Ryk protein from the result of step (b).

21. An antibody against a protein according to claim 8.

22. An antibody against a protein according to claim 27.

23. An antibody having an amino acid sequence identified in SEQ ID NO: 22.

24. A method of modulating angiogenesis at a site, the method comprising causing an effective amount of a composition comprising the antibody of claim 27 to be supplied to the site.

25. A method for diagnosing a disease or medical condition or susceptibility to a disease or medical condition in a mammal, the disease or medical condition related to inadequate or excess expression of a variant a Ryk protein, wherein the variant Ryk protein has an amino acid sequence selected from the group consisting essentially of SEQ ID NO: 1, SEQ ID NO: 2 and SEQ ID NO: 3, the method comprising the steps:
(a) determining the level of expression of said protein in a sample obtained from the mammal; and
(b) comparing the level of expression of said protein against a standard to make a diagnosis.

26. The method of claim 25, wherein the medical condition is selected from the group consisting of cancer, metastasis, diabetic retinopathy, macular degeneration, cardiovascular disease, a wound, pregnancy, and a clinical condition involving angiogenesis in the reproductive system.

27. A protein characterized by having a deduced amino acid sequence selected from the group consisting essentially of SEQ ID NO: 1, SEQ ID NO: 2 and SEQ ID NO: 3.


SEQUENCE LISTING (23 sequences)

SEQ ID NO: 1 - protein, 191 aa, HOMO SAPIENS
APAPRPPELQSASAGPSVSLYLSEDEVRRLIGLDAELYYVRNDLISHYALSFNLLVPSETNFLHFTWHAKSKVEYKLGFQVDNVLAMDMPQVNISVQGEVPRTLSVFRVELSCTGKVDSEVMILMQLNLTVNSSKNFTVLNFKRRKMCYKKLEEVKTSALDKNTSRTIYDPVHAAPTTSTRVFYISVGVCC

SEQ ID NO: 2 - protein, 181 aa, HOMO SAPIENS
APAPRPPELQSASAGPSVSLYLSEDEVRRLIGLDAELYYVRNDLISHYALSFNLLVPSETNFLHFTWHAKSKVEYKLGFQVDNVLAMDMPQVNISVQGEVPRTLSVFRVELSCTGKVDSEVMILMQLNLTVNSSKNFTALNFKRRKMCYKKLEEVKTSALDKNTSRTIYDPVHAAPTTSTR

SEQ ID NO: 3 - protein, 180 aa, HOMO SAPIENS
MRGAARLGRPGRSCLPGARGLRAPPPPPLLLLLALLPLLPAPGAAASVSLYLSEDEVRRLIGLDAELYYVRNDLISHYALSFNLLVPSETNFLHFTWHAKSKVEYKLGFQVDNVLAMDMPQVNISVQGEVPRTLSVFRVELSCTGKVDSEVMILMQLNLTVNSSKNFTVLNFKRRKMCYK

SEQ ID NO: 4 - protein, 233 aa, ARTIFICIAL
  Fc tag
AAADDDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKCQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 46 aa, HOMO SAPIENS
MRGAARLGRPGRSCLPGARGLRAPPPPPLLLLLALLPLLPAPGAAA

SEQ ID NO: 6 - protein, 607 aa, HOMO SAPIENS
MRGAARLGRPGRSCLPGARGLRAPPPPPLLLLLALLPLLPAPGAAAAPAPRPPELQSASAGPSVSLYLSEDEVRRLIGLDAELYYVRNDLISHYALSFNLLVPSETNFLHFTWHAKSKVEYKLGFQVDNVLAMDMPQVNISVQGEVPRTLSVFRVELSCTGKVDSEVMILMQLNLTVNSSKNFTALNFKRRKMCYKKLEEVKTSALDKNTSRTIYDPVHAAPTTSTRVFYISVGVCCAVIFLVAIILAVLHLHNMKRIELDDSISASSSSQGLSQPSTQTTQYLRADTPNNATPITSYPTLRIEKNDLRSVTLLEAKGKVKDIAISRERITLKDVLQEGTFGRIFHGILIDEKDPNKEKQAFVKTVKDQASEIQVTMMLTESCKLRGLHHRNLLPITHVCIEEGEKPMVILPYMNWGNLKLFLRQCKLVEANNPQAISQQDLVHMAIQIACGMSYLARREVIHKDLAARNCVIDDTLQVKITDNALSRDLFPMDYHCLGDNENRPVRWMALESLVNNEFSSASDVWAFGVTLWELMTLGQTPYVDIDPFEMAAYLKDGYRIAQPINCPDELFAVMACCWALDPEERPKFQQLVQCLTEFHAALGAYV

SEQ ID NO: 7 - protein, 414 aa, ARTIFICIAL
  Fusion of human protein fragment and Fc tag
APAPRPPELQSASAGPSVSLYLSEDEVRRLIGLDAELYYVRNDLISHYALSFNLLVPSETNFLHFTWHAKSKVEYKLGFQVDNVLAMDMPQVNISVQGEVPRTLSVFRVELSCTGKVDSEVMILMQLNLTVNSSKNFTALNFKRRKMCYKKLEEVKTSALDKNTSRTIYDPVHAAPTTSTRAAADDDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKCQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 218 aa, ARTIFICIAL
  Fusion of human protein fragment and myc-his tag
APAPRPPELQSASAGPSVSLYLSEDEVRRLIGLDAELYYVRNDLISHYALSFNLLVPSETNFLHFTWHAKSKVEYKLGFQVDNVLAMDMPQVNISVQGEVPRTLSVFRVELSCTGKVDSEVMILMQLNLTVNSSKNFTALNFKRRKMCYKKLEEVKTSALDKNTSRTIYDPVHAAPTTSTRVFYISVGVCCLESRGPEQKLISEEDLNSAVDHHHHHH

SEQ ID NO: 9 - protein, 408 aa, ARTIFICIAL
  Fusion of human protein fragment and Fc tag
MRGAARLGRPGRSCLPGARGLRAPPPPPLLLLLALLPLLPAPGAAASVSLYLSEDEVRRLIGLDAELYYVRNDLISHYALSFNLLVPSETNFLHFTWHAKSKVEYKLGFQVDNVLAMDMPQVNISVQGEVPRTLSVFRVELSCTGKVDSEVMILMQLNLTVNSSKNFTVLNFKRRKMCYKSRGPFECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 204 aa, ARTIFICIAL
  Fusion of human protein fragment and V5-his tag
MRGAARLGRPGRSCLPGARGLRAPPPPPLLLLLALLPLLPAPGAAASVSLYLSEDEVRRLIGLDAELYYVRNDLISHYALSFNLLVPSETNFLHFTWHAKSKVEYKLGFQVDNVLAMDMPQVNISVQGEVPRTLSVFRVELSCTGKVDSEVMILMQLNLTVNSSKNFTVLNFKRRKMCYKKGVEVKPILNPLLGLDSTHHHHHH

SEQ ID NO: 11 - protein, 51 aa, ARTIFICIAL
  Illustrative random sequence
RTPSDKPVAHVANPQLQWLNRRANALLANGVERDNQLVVEGLYLIYSQVLF

SEQ ID NO: 12 - protein, 52 aa, ARTIFICIAL
  Illustrative random sequence
RAPFKKSWAYLQVAKHKLSWNKDGILHGVRYQDGNLVIQFPGLYFIICQLQF

SEQ ID NO: 13 - protein, 8 aa, ARTIFICIAL
  FLAG marker peptide
DYKDDDDK

SEQ ID NO: 14 - DNA, 711 bp, HOMO SAPIENS
atgcgtggggcggcgcggctggggcggccgggccggagttgcctcccgggggcccgcggcctgagggccccgccgccgccgccgctgctgcttctgcttgcgctgttgccgctgctgcccgcgcctggcgctgccgccgcccccgccccgcggcccccggagctgcagtcggcttccgcggggcccagcgtgagtctctacctgagcgaggacgaggtgcgccggctgatcggtcttgatgcagaactttattatgtgagaaatgaccttattagtcactacgctctatcctttaatctgttagtacccagtgagacaaatttcctgcacttcacctggcatgcgaagtccaaggttgaatataagctgggattccaagtggacaatgttttggcaatggatatgccccaggtcaacatttctgttcagggggaagttccacgcactttatcagtgtttcgggtagagctttcctgtactggcaaagtagattctgaagttatgatactaatgcagctcaacttgacagtaaattcttcaaaaaattttaccgtcttaaattttaaacgaaggaaaatgtgctacaaaaaacttgaagaagtaaaaacttcagccttggacaaaaacactagcagaactatttatgatcctgtacatgcagctccaaccacttctacgcgtgtgttttatattagtgtaggggtttgttgt

SEQ ID NO: 15 - DNA, 681 bp, HOMO SAPIENS
atgcgtggggcggcgcggctggggcggccgggccggagttgcctcccgggggcccgcggcctgagggccccgccgccgccgccgctgctgcttctgcttgcgctgttgccgctgctgcccgcgcctggcgctgccgccgcccccgccccgcggcccccggagctgcagtcggcttccgcggggcccagcgtgagtctctacctgagcgaggacgaggtgcgccggctgatcggtcttgatgcagaactttattatgtgagaaatgaccttattagtcactacgctctatcctttaatctgttagtacccagtgagacaaatttcctgcacttcacctggcatgcgaagtccaaggttgaatataagctgggattccaagtggacaatgttttggcaatggatatgccccaggtcaacatttctgttcagggggaagttccacgcactttatcagtgtttcgggtagagctttcctgtactggcaaagtagattctgaagttatgatactaatgcagctcaacttgacagtaaattcttcaaaaaattttaccgtcttaaattttaaacgaaggaaaatgtgctacaaaaaacttgaagaagtaaaaacttcagccttggacaaaaacactagcagaactatttatgatcctgtacatgcagctccaaccacttctacgcgt

SEQ ID NO: 16 - DNA, 543 bp, HOMO SAPIENS
atgcgtggggcggcgcggctggggcggccgggccggagttgcctcccgggggcccgcggcctgagggccccgccgccgccgccgctgctgcttctgcttgcgctgttgccgctgctgcccgcgcctggcgctgccgccagcgtgagtctctacctgagcgaggacgaggtgcgccggctgatcggtcttgatgcagaactttattatgtgagaaatgaccttattagtcactacgctctatcctttaatctgttagtacccagtgagacaaatttcctgcacttcacctggcatgcgaagtccaaggttgaatataagctgggattccaagtggacaatgttttggcaatggatatgccccaggtcaacatttctgttcagggggaagttccacgcactttatcagtgtttcgggtagagctttcctgtactggcaaagtagattctgaagttatgatactaatgcagctcaacttgacagtaaattcttcaaaaaattttaccgtcttaaattttaaacgaaggaaaatgtgctacaaatga

SEQ ID NO: 17 - DNA, 1383 bp, ARTIFICIAL
  Fusion of human cDNA and Fc tag coding sequence
atgcgtggggcggcgcggctggggcggccgggccggagttgcctcccgggggcccgcggcctgagggccccgccgccgccgccgctgctgcttctgcttgcgctgttgccgctgctgcccgcgcctggcgctgccgccgcccccgccccgcggcccccggagctgcagtcggcttccgcggggcccagcgtgagtctctacctgagcgaggacgaggtgcgccggctgatcggtcttgatgcagaactttattatgtgagaaatgaccttattagtcactacgctctatcctttaatctgttagtacccagtgagacaaatttcctgcacttcacctggcatgcgaagtccaaggttgaatataagctgggattccaagtggacaatgttttggcaatggatatgccccaggtcaacatttctgttcagggggaagttccacgcactttatcagtgtttcgggtagagctttcctgtactggcaaagtagattctgaagttatgatactaatgcagctcaacttgacagtaaattcttcaaaaaattttaccgtcttaaattttaaacgaaggaaaatgtgctacaaaaaacttgaagaagtaaaaacttcagccttggacaaaaacactagcagaactatttatgatcctgtacatgcagctccaaccacttctacgcgtgcggccgcagatgatgatgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 18 - DNA, 795 bp, ARTIFICIAL
  Fusion of human cDNA and myc-his tag coding sequence
atgcgtggggcggcgcggctggggcggccgggccggagttgcctcccgggggcccgcggcctgagggccccgccgccgccgccgctgctgcttctgcttgcgctgttgccgctgctgcccgcgcctggcgctgccgccgcccccgccccgcggcccccggagctgcagtcggcttccgcggggcccagcgtgagtctctacctgagcgaggacgaggtgcgccggctgatcggtcttgatgcagaactttattatgtgagaaatgaccttattagtcactacgctctatcctttaatctgttagtacccagtgagacaaatttcctgcacttcacctggcatgcgaagtccaaggttgaatataagctgggattccaagtggacaatgttttggcaatggatatgccccaggtcaacatttctgttcagggggaagttccacgcactttatcagtgtttcgggtagagctttcctgtactggcaaagtagattctgaagttatgatactaatgcagctcaacttgacagtaaattcttcaaaaaattttaccgtcttaaattttaaacgaaggaaaatgtgctacaaaaaacttgaagaagtaaaaacttcagccttggacaaaaacactagcagaactatttatgatcctgtacatgcagctccaaccacttctacgcgtgtgttttatattagtgtaggggtttgttgtctcgagtctagagggcccgaacaaaaactcatctcagaagaggatctgaatagcgccgtcgaccatcatcatcatcatcattga

SEQ ID NO: 19 - DNA, 1271 bp, ARTIFICIAL
  Fusion of human cDNA and Fc tag coding sequence
atgcgtggggcggcgcggctggggcggccgggccggagttgcctcccgggggcccgcggcctgagggccccgccgccgccgccgctgctgcttctgcttgcgctgttgccgctgctgcccgcgcctggcgctgccgccagcgtgagtctctacctgagcgaggacgaggtgcgccggctgatcggtcttgatgcagaactttattatgtgagaaatgaccttattagtcactacgctctatcctttaatctgttagtacccagtgagacaaatttcctgcacttcacctggcatgcgaagtccaaggttgaatataagctgggattccaagtggacaatgttttggcaatggatatgccccaggtcaacatttctgttcagggggaagttccacgcactttatcagtgtttcgggtagagctttcctgtactggcaaagtagattctgaagttatgatactaatgcagctcaacttgacagtaaattcttcaaaaaattttaccgtcttaaattttaaacgaaggaaaatgtgctacaaatctagagggcccttcgaatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaactagagacgcgtgtcgacgcggccgcgataaacggccggcaag

SEQ ID NO: 20 - DNA, 615 bp, ARTIFICIAL
  Fusion of human cDNA and V5-his tag coding sequence
atgcgtggggcggcgcggctggggcggccgggccggagttgcctcccgggggcccgcggcctgagggccccgccgccgccgccgctgctgcttctgcttgcgctgttgccgctgctgcccgcgcctggcgctgccgccagcgtgagtctctacctgagcgaggacgaggtgcgccggctgatcggtcttgatgcagaactttattatgtgagaaatgaccttattagtcactacgctctatcctttaatctgttagtacccagtgagacaaatttcctgcacttcacctggcatgcgaagtccaaggttgaatataagctgggattccaagtggacaatgttttggcaatggatatgccccaggtcaacatttctgttcagggggaagttccacgcactttatcagtgtttcgggtagagctttcctgtactggcaaagtagattctgaagttatgatactaatgcagctcaacttgacagtaaattcttcaaaaaattttaccgtcttaaattttaaacgaaggaaaatgtgctacaaaaagggcgtcgaggttaagcctatccttaaccctctcctcggtctcgattctacgcatcatcaccatcaccattga

SEQ ID NO: 21 - protein, 27 aa, ARTIFICIAL
  Myc-his tag peptide sequence
LESRGPEQKLISEEDLNSAVDHHHHHH

SEQ ID NO: 22 - protein, 271 aa, ARTIFICIAL
  Amino acid sequence for scFv 1b4 antibody
DYKDEVQLVQSGAEVKKPGSSVKVSCKASGGTFSSYAISWVRQAPGQGLEWMGGIIPIFGTANYAQKFQGRVTITADESTSTAYMELSSLRSEDTAVYYCARSYYDWFDYWGQGTLVTVSSAGGGSGGGGSGGGGSGGGGSDIVLTQPPSVSGAPGQRVTISCSGSSSNIGSNYVSWYQQLPGTAPKLLIYDNNQRPSGVPDRFSGSKSGTSASLAITGLQSEDEADYYCQSWDSIAQIVFGGGTKLTVLGQEFDYKDDDDKGAPHHHHHH

SEQ ID NO: 23 - DNA, 876 bp, ARTIFICIAL
  Nucleotide sequence encoding scFv 1b4 antibody
atgaaacaaagcactattgcactggcactcttaccgttgctcttcacccctgttaccaaagccgactacaaagatgaagtgcaattggttcagtctggcgcggaagtgaaaaaaccgggcagcagcgtgaaagtgagctgcaaagcctccggaggcacttttagcagctatgcgattagctgggtgcgccaagcccctgggcagggtctcgagtggatgggcggcattattccgatttttggcacggcgaactacgcgcagaagtttcagggccgggtgaccattaccgcggatgaaagcaccagcaccgcgtatatggaactgagcagcctgcgtagcgaagatacggccgtgtattattgcgcgcgttcttattatgattggtttgattattggggccaaggcaccctggtgacggttagctcagcgggtggcggttctggcggcggtgggagcggtggcggtggttctggcggtggtggttccgatatcgtgctgacccagccgccttcagtgagtggcgcaccaggtcagcgtgtgaccatctcgtgtagcggcagcagcagcaacattggcagcaactatgtgagctggtaccagcagttgcccgggacggcgccgaaactgctgatttatgataacaaccagcgtccctcaggcgtgccggatcgttttagcggatccaaaagcggcaccagcgcgagccttgcgattacgggcctgcaaagcgaagacgaagcggattattattgccagagctgggactctattgctcagattgtgtttggcggcggcacgaagttaaccgttcttggccaggaattcgactataaagatgacgatgacaaaggcgcgccgcaccatcatcaccatcac